(19) United States
(12) Patent Application Publication
(10) Pub. No.: US 2011/0172648 A1
(43) Pub. Date: Jul. 14, 2011

(54) TOOL FOR MINIMALLY INVASIVE SURGERY AND METHOD FOR USING THE SAME

(76) Inventor: Chang Wook Jeong, Seoul (KR)

(21) Appl. No.: 13/063,918

(22) PCT Filed: Sep. 9, 2009
(86) PCT No.: PCT/KR09/05104

§ 371 Date: Mar. 14, 2011

(51) Int. Cl. A61B 17/00

(52) U.S. Cl. 606/1

(57) ABSTRACT

The present invention relates to an easy-to-control tool for minimally invasive surgery and a method for using the same. In accordance with an aspect of the present invention, there is provided a tool for minimally invasive surgery and a method for using the same comprising, a main shaft, a first control shaft and a second control shaft positioned in sequence from one end of the main shaft, a first actuating shaft and a second actuating shaft positioned in sequence from the other end of the main shaft, an adjustment handle positioned around one end of the second control shaft, an end effector positioned around one end of the second actuating shaft, a pitch control part positioned around one position of the positions between the main shaft and the first control shaft, between the first control shaft and the second control shaft, and between the second control shaft and the adjustment handle, for transferring a motion of the adjustment handle in a pitch direction to the end effector, a first yaw control part and a second yaw control part positioned around the other positions of the positions between the main shaft and the first control shaft, between the first control shaft and the second control shaft, and between the second control shaft and the adjustment handle, respectively, for transferring a motion of the adjustment handle in a yaw direction to the end effector, a pitch actuating part positioned around one position of the positions between the main shaft and the first actuating shaft, between the first actuating shaft and the second actuating shaft, and between the second actuating shaft and the end effector, a first yaw actuating part and a second yaw actuating part positioned around the other positions of the positions between the main shaft and the first actuating shaft, between the first actuating shaft and the second actuating shaft, and between the second actuating shaft and the end effector, respectively, a first pitch cable and a second pitch cable for transferring motions from the pitch control part, the first yaw control part, and the second yaw control part to the pitch actuating part, the first yaw actuating part, and the second yaw actuating part, respectively, and a yaw cable for transferring a motion from the first yaw control part to the first yaw actuating part with the first pitch cable and the second pitch cable.

### FIELD OF THE INVENTION

The present invention relates to an easy-to-control tool for minimally invasive surgery and a method for using the same, and more specifically, to a tool for minimally invasive surgery, which performs minimally invasive surgery in a dexterous and convenient manner by actuating an end effector through the control of an adjustment handle, and a method for using the same.

### BACKGROUND OF THE INVENTION

Minimally invasive surgery is a surgical approach that involves use of instruments inserted through several tiny incision openings to perform a surgery causing minimal tissue trauma.

This minimally invasive surgery relatively reduces changes in metabolism of the patient in the period of post-surgical care, so it is beneficial to rapid recovery of the patient. Therefore, using such minimally invasive surgery shortens length of a hospital stay of the patient after the surgery and allows patients to return to normal physical activities more quickly. In addition, minimally invasive surgery causes less pain and reduces scar to patients after surgery.

The most general form of the minimally invasive surgery is endoscopy. Among them, a laparoscopy that involves minimally-invasive inspection and operation inside abdominal cavity is known as the most general form of endoscopy. To operate the standard laparoscopic surgery, an abdomen of the patient is insufflated with gas, and small incisions (about ½ inch or less) are formed for use as an entrance of a tool for the laparoscopic surgery, through which a trocar is inserted. In general, laparoscopic surgical tools include a laparoscope (for observation of a surgical site) and other working tools. Here, the working tools are similar in structure to the conventional tools used for small incision surgery, except that the end effector or working end of each tool is separated from its handle by an elongated shaft. For instance, working tools may include a clamp, a grasper, scissors, a stapler, needle holder, and so forth. To perform the surgery, a user, such as a surgeon, puts the working tool into a surgical site through the trocar, and manipulates it from the outside of abdominal cavity. Then, the surgeon monitors the procedure of the surgery through a monitor that displays the image of the surgical site that is taken by the laparoscope. The endoscopic approach similar to this is broadly used in retroperitoneoscopy, pelviscopy, arthroscopy, cisternoscopy, sinuscopy, hysteroscopy, nephroscopy, cystoscopy, urethroscopy, pyeloscopy, and so on.

Although this minimally invasive surgery has a number of advantages, it has shortcomings in the difficulty of approaching the conventional minimally invasive surgical tools to a surgical site and the inconvenient or complicate manipulation of such tools because of an end effector connected to a rigid and long shaft. Particularly, since the traditional end effector has no bending portion like a joint, it is difficult to perform a dexterous handling required for surgery.

Moreover, when a surgical site is located behind a specific organ, the conventional minimally invasive surgical tools cannot even approach there.

Also, traditionally, many surgical tools were often used together even for minimally invasive surgery, and because of that many incisions were formed in a patient's body. An attempt to solve such a problem has been made by proposing the idea of forming only one incision and then inserting a trocar into the incision for surgery, but unfortunately there is no suitable tool for supporting the idea.

In view of the foregoing, the present inventor noticed that all the problems mentioned above are, after all, the main impediment to the wide expansion of minimally invasive surgery.

### SUMMARY OF THE INVENTION

The present invention is directed to solve all of the problems mentioned above.

Another object of the present invention is to provide a tool for minimally invasive surgery, which has an end effector of high-degree-of-freedom motion.

Still another object of the present invention is to provide a tool for minimally invasive surgery, which operates in a dexterous manner with relatively simple drive control elements.

Still another object of the present invention is to provide a tool for minimally invasive surgery, which easily accesses to areas that are hidden by specific organs, including plural joint portions, for surgery.

Still another object of the present invention is to provide a surgical tool for achieving a minimally invasive surgery in a dexterous and convenient manner with the least number of incisions in a patient's body, most preferably, with only one incision.

Still another object of the present invention is to provide a tool for minimally invasive surgery, which is more technically advanced than the minimally invasive surgical tools that are disclosed in Korean Patent Application Nos. 2008-51248 and 2008-61894 filed by the same inventor.

Yet another object of the present invention is to provide a novel method for using a tool for minimally invasive surgery in accordance with the present invention.

In accordance with an aspect of the present invention, there is provided a tool for minimally invasive surgery comprising, a main shaft, a first control shaft and a second control shaft positioned in sequence from one end of the main shaft, a first actuating shaft and a second actuating shaft positioned in sequence from the other end of the main shaft, an adjustment handle positioned around one end of the second control shaft, an end effector positioned around one end of the second actuating shaft, a pitch control part positioned around one position of the positions between the main shaft and the first control shaft, between the first control shaft and the second control shaft, and between the second control shaft and the adjustment handle, for transferring a motion of the adjustment handle in a pitch direction to the end effector, a first yaw control part and a second yaw control part positioned around the other positions of the positions between the main shaft and the first control shaft, between the first control shaft and the second control shaft, and between the second control shaft and the adjustment handle, respectively, for transferring a motion of the adjustment handle in a yaw direction to the end effector, a pitch actuating part positioned around one position of the positions between the main shaft and the first actuating shaft, between the first actuating shaft and the second actuating shaft, and between the second actuating shaft and the end effector, a first yaw actuating part and a second yaw actuating part positioned around the other positions of the positions between the main shaft and the first actuating shaft, between the first actuating shaft and the second actuating shaft, and between the second actuating shaft and the end effector, respectively, a first pitch cable and a second pitch cable for transferring motions from the pitch control part, the first yaw control part, and the second yaw control part to the pitch actuating part, the first yaw actuating part, and the second yaw actuating part, respectively, and a yaw cable for transferring a motion from the first yaw control part to the first yaw actuating part with the first pitch cable and the second pitch cable.

In accordance with another aspect of the present invention, there is provided a tool for minimally invasive surgery comprising, a main shaft, an adjustment handle positioned around one end of the main shaft, a first actuating shaft and a second actuating shaft positioned in sequence from the other end of the main shaft, an end effector positioned around one end of the second actuating shaft, a connection part positioned between the main shaft and the adjustment handle for transferring motions of the adjustment handle in a pitch direction and a yaw direction to the end effector, a pitch actuating part positioned around one position of the positions between the main shaft and the first actuating shaft, between the first actuating shaft and the second actuating shaft, and between the second actuating shaft and the end effector, a first yaw actuating part and a second yaw actuating part positioned around the other positions of the positions between the main shaft and the first actuating shaft, between the first actuating shaft and the second actuating shaft, and between the second actuating shaft and the end effector, respectively, a first pitch cable and a second pitch cable for transferring motions from the connection part to the pitch actuating part, the first yaw actuating part, and second yaw actuating part, and a yaw cable for transferring a motion from the connection part to the first yaw actuating part with the first pitch cable and the second pitch cable.

In accordance with yet another aspect of the present invention, there is provided a tool for minimally invasive surgery comprising, a main shaft, a first control shaft and a second control shaft positioned in sequence from one end of the main shaft, a first actuating shaft and a second actuating shaft positioned in sequence from the other end of the main shaft, an adjustment handle positioned around one end of the second control shaft, an end effector positioned around one end of the second actuating shaft, a first connection part positioned between the second control shaft and the adjustment handle for transferring motions of the adjustment handle in a pitch direction and a yaw direction to the end effector, a first yaw control part and a second yaw control part positioned between the main shaft and the first control shaft, and between the first control shaft and the second control shaft, respectively, for transferring a motion of the adjustment handle in a yaw direction to the end effector, a second connection part positioned between the second actuating shaft and the end effector, a first yaw actuating part and a second yaw actuating part positioned between the main shaft and the first actuating shaft, and between the first actuating shaft and second actuating shaft, a first pitch cable and a second pitch cable for transferring motions from the first connection part, the first yaw control part, and the second yaw control part to the second connection part, the first yaw actuating part, and the yaw actuating part, respectively, a first yaw cable for transferring a motion from the second yaw control part to the second yaw actuating part with the first pitch cable and the second pitch cable, and a second yaw cable for transferring a motion from the first yaw control part to the first yaw actuating part with the first pitch cable, the second pitch cable, and the first yaw cable.

In accordance with still yet another aspect of the present invention, there is provided a tool for minimally invasive surgery comprising, a main shaft, a first actuating shaft and a second actuating shaft positioned in sequence from one end of the main shaft, a controller positioned around the other end of the main shaft, an end effector positioned around one end of the second actuating shaft, a pitch actuating part positioned around one position of the positions between the main shaft and the first actuating shaft, between the first actuating shaft and the second actuating shaft, and between the second actuating shaft and the end effector, a first yaw actuating part and a second yaw actuating part positioned around the other positions of the positions between the main shaft and the first actuating shaft, between the first actuating shaft and the second actuating shaft, and between the second actuating shaft and the end effector, respectively, and a first pitch cable, a second pitch cable, and a yaw cable for controlling operations of the pitch actuating part, the first yaw actuating part, and the second yaw actuating part, wherein the controller comprises a pitch control module for controlling the pitch actuating part, a first yaw control module for controlling the first yaw actuating part, and a second yaw control module for controlling the second yaw actuating part.

In accordance with still yet another aspect of the present invention, there are provided various methods of using the tools for minimally invasive surgery according to the present invention.

### BRIEF DESCRIPTION OF THE DRAWINGS

The above and other objects and features of the present invention will become apparent from the following description of preferred embodiments, given in conjunction with the accompanying drawings, in which:

FIG. 1 is a perspective view showing the outer appearance of a tool for minimally invasive surgery in accordance with a first embodiment of the present invention;

FIG. 2 is an exploded perspective view showing a connection between a second control shaft and an adjustment handle in accordance with the first embodiment of the present invention;

FIG. 3 is a detailed view of ‘a’ portion in FIG. 1;

FIG. 4 is an exploded perspective view showing a configuration of a first yaw control part in accordance with the first embodiment of the present invention;

FIGS. 5, 6 and 7 show a configuration of a first control main body seen from different angles, the first control main body being used for the first yaw control part in accordance with the first embodiment of the present invention;

FIG. 8 is a detailed view of ‘b’ portion in FIG. 1;

FIG. 9 is an exploded perspective view showing a configuration of a second yaw control part depicted in FIG. 8;

FIG. 10 is a detailed view of ‘c’ portion in FIG. 1;

FIG. 11 is an exploded perspective view showing a configuration of a first yaw actuating part in accordance with the first embodiment of the present invention;

FIG. 12 is a detailed view of ‘d’ portion in FIG. 1;

FIG. 13 is an exploded perspective view showing a configuration of a second yaw actuating part in accordance with the first embodiment of the present invention;

FIG. 14 is a detailed view of ‘e’ portion in FIG. 1;

FIG. 15 is an exploded perspective view showing how an end effector is connected to a second actuating shaft in accordance with the first embodiment of the present invention;

FIG. 16 shows an example of how yaw cable is wound around a first control main body and a first actuating main body that constitute the first yaw control part and the first yaw actuating part, respectively, in accordance with the first embodiment of the present invention;

FIG. 17 shows an example of how cables are connected in the second yaw control part in accordance with the first embodiment of the present invention;

FIG. 18 shows an example of how cables are connected in the first yaw control part in accordance with the first embodiment of the present invention;

FIG. 19 shows a connection state between first and second pitch cables in the first yaw actuating part in accordance with the first embodiment of the present invention;

FIG. 20 shows a connection state between first and second pitch cables in the second yaw actuating part in accordance with the first embodiment of the present invention;

FIGS. 21, 22 and 23 show an operational state of a tool for minimally invasive surgery in accordance with the first embodiment of the present invention;

FIGS. 24 and 25 show a perspective view showing a connection between a second control shaft and an adjustment handle of a tool for minimally invasive surgery in accordance with a second embodiment of the present invention, in which FIG. 24 shows a first rod being extended and FIG. 25 shows a second rod being extended;

FIG. 26 is a perspective view showing the outer appearance of a tool for minimally invasive surgery in accordance with a third embodiment of the present invention;

FIG. 27 is a detailed view of ‘b’ portion in FIG. 26;

FIG. 28 is a detailed view of ‘a’ portion in FIG. 26;

FIG. 29 is a perspective view showing the outer appearance of a tool for minimally invasive surgery in accordance with a fourth embodiment of the present invention;

FIG. 30 is a detailed view of ‘a’ portion in FIG. 29;

FIG. 31 is an exploded perspective view showing a configuration of a connection part between a shaft and an adjustment handle in accordance with the fourth embodiment of the present invention;

FIG. 32 shows an example of how yaw cable is connected in accordance with the fourth embodiment of the present invention;

FIG. 33 and FIG. 34 show an operational state of the tool for minimally invasive surgery in accordance with the fourth embodiment of the present invention;

FIG. 35 is a perspective view showing the outer appearance of a tool for minimally invasive surgery in accordance with a fifth embodiment of the present invention;

FIGS. 36 and 37 show a detailed view of ‘a’ portion in FIG. 35, which show a configuration of a first yaw control part in accordance with the fifth embodiment of the present invention seen from different angles;

FIG. 38 is an exploded perspective view showing a configuration of the first yaw control part in accordance with the fifth embodiment of the present invention;

FIGS. 39 and 40 show a detailed view of ‘b’ portion in FIG. 18, which show a configuration of a second yaw control part in accordance with the fifth embodiment of the present invention seen from different angles;

FIG. 41 is an exploded perspective view showing a configuration of the second yaw control part in accordance with the fifth embodiment of the present invention;

FIG. 42 is a detailed view of ‘c’ portion in FIG. 18;

FIG. 43 is an exploded perspective view showing a configuration of a first yaw actuating part in accordance with the fifth embodiment of the present invention;

FIG. 44 is a detailed view of ‘d’ portion in FIG. 18;

FIG. 45 is an exploded perspective view showing a configuration of a second yaw actuating part in accordance with the fifth embodiment of the present invention;

FIGS. 46 and 47 show an operational state of the tool for minimally invasive surgery in accordance with the fifth embodiment of the present invention;

FIG. 48 is a perspective view showing the outer appearance of a tool for minimally invasive surgery in accordance with a first application example of the present invention; and

FIGS. 49, 50 and 51 show a method for using a tool for minimally invasive surgery in accordance with the present invention.

### DETAILED DESCRIPTION OF THE PREFERRED EMBODIMENT

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the invention, although different, are not necessarily mutually exclusive. The following detailed description is, therefore, not to be taken in a limiting sense, and the scope of the present invention is defined only by the appended claims that should be appropriately interpreted along with the full range of equivalents to which the claims are entitled.

Hereinafter, preferred embodiments of the present invention will be explained in detail with reference to the accompanying drawing so that the invention can easily be practiced by those skilled in the art.

### Embodiment 1

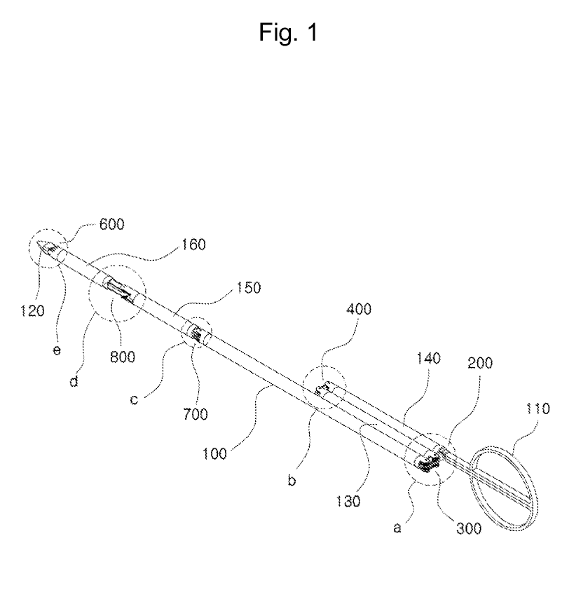

FIG. 1 is a perspective view showing the outer appearance of a tool 1 for minimally invasive surgery, in accordance with a first embodiment of the present invention.

Referring to FIG. 1, the tool 1 for minimally invasive surgery of this embodiment includes a shaft 100 (i.e., main shaft), an adjustment handle 110, an end effector 120, first and second control shafts 130 and 140, first and second actuating shafts 150 and 160, a pitch control part 200, first and second yaw control parts 300 and 400, a pitch actuating part 600, and first and second yaw actuating parts 700 and 800.

First, as shown in FIG. 1, there is the main shaft 100, and the first and second control shafts 130 and 140 are positioned in sequence from one end of the main shaft 100, and the first and the second actuating shafts 150 and 160 are positioned in sequence from the other end of the main shaft 100. At least part of the shafts have, if necessary, one or plural spaces (for example, tube-shape, lotus root-shape or spiral-shape space(s))(not shown) inside.

In addition, the adjustment handle 110 is positioned around one end of the second control shaft 140, and the end effector 120 is positioned around one end of the second actuating shaft 160, as shown.

Hereinafter, a configuration of the tool 1 for minimally invasive surgery in accordance with the first embodiment of the present invention will be explained in further detail, with reference to the drawings.

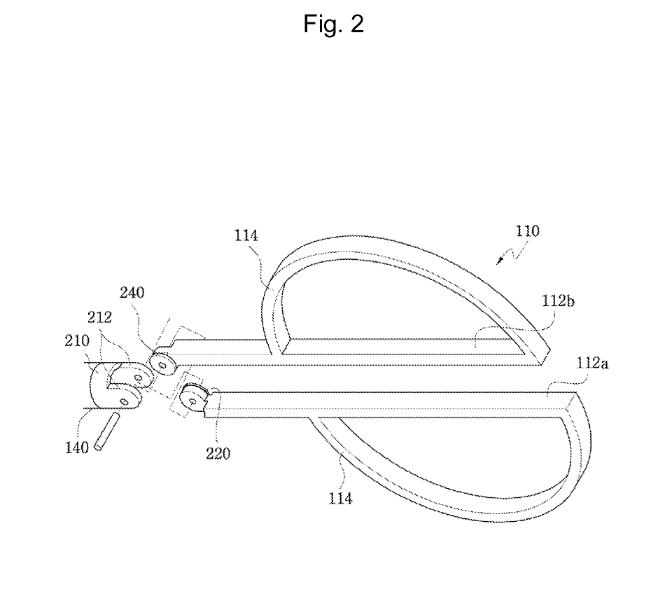

FIG. 2 is an exploded perspective view showing a connection between a second control shaft 140 and an adjustment handle 110 in accordance with the first embodiment of the present invention. As shown, the adjustment handle 110 includes first and second rods 112a and 112b each of which having one end connected to a rotation axis and semi-circular enclosures 114 symmetric with each other.

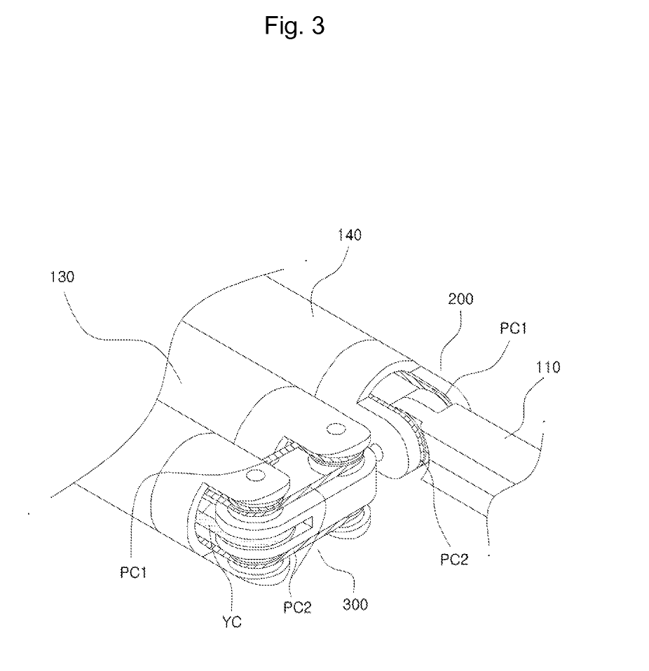

Disposed on the rotation axis to which the first and second rods 112a and 112b of the adjustment handle 110 are connected first and second pitch cable pulleys 220 and 240 of a pitch control part 200 (to be described) as shown in FIG. 2. Here, the rotation axis may be placed across a connection ring 210 which is connected to one end of a second control shaft 140 and has a pair of connection ends 212. More details on this will now be provided with reference to FIG. 3A.

FIG. 3 is a detailed view of ‘a’ portion in FIG. 1, which shows that the second control shaft 140 and the adjustment handle 110 are connected to each other by the pitch control part 200, and a main shaft 100 and a first control shaft 130 are connected to each other by a first yaw control part 300, in accordance with the first embodiment of the present invention.

Referring to FIG. 3, the pitch control part 200 may include the first and the second pitch cable pulleys 220 and 240 as noted earlier. Here, the first pitch cable pulley 220 and the second pitch cable pulley 240 have substantially the same diameter, and they also have substantially the same width as first and second pitch cables PC1 and PC2. The first and the second pitch cable pulleys 220 and 240 are operationally coupled to the first and the second rods 112a and 112b that constitute the adjustment handle 110 to operate the first and the second pitch cables PC1 and PC2, as will be described later.

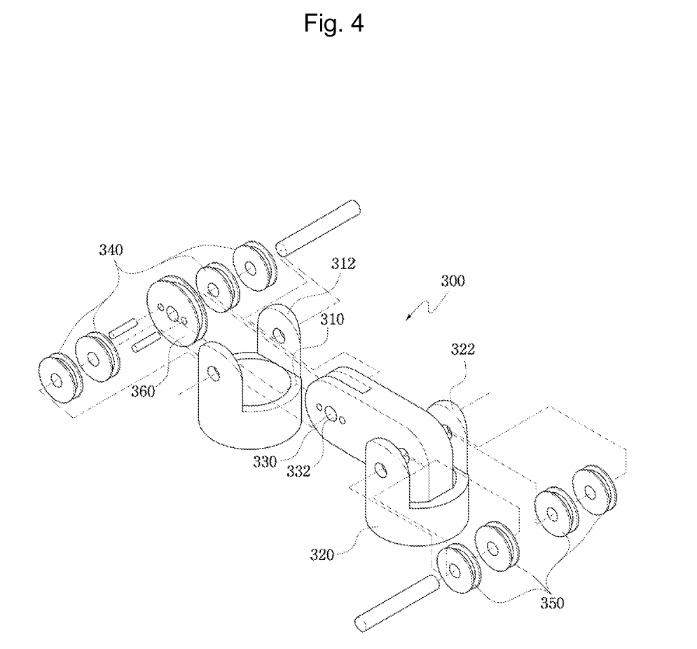

A configuration of the first yaw control part 300 in accordance with the first embodiment of the present invention will now be explained with reference to FIG. 3 and to its exploded perspective view in FIG. 4.

Referring to FIG. 4, the first yaw control part 300 includes first and second connection rings 310 and 320 each of which has a circular ring form (although connection rings throughout the specification are preferably in a ring or similar shape to facilitate the operation of cables, they are not necessarily limited to such a shape). The first and the second connection rings 310 and 320 can be connected to the main shaft 100 and the first control shaft 130, respectively, or positioned near both ends of a first control main body 330 (to be described later). In addition, a pair of first connection ends 312 is formed on the first connection ring 310, and a pair of second connection ends 322 is formed on the second connection ring 320. Preferably, the first and the second connection end pairs 312 and 322 are formed in a manner that they are substantially parallel to each other with respect to central axes of the first and the second connection rings 310 and 320, respectively.

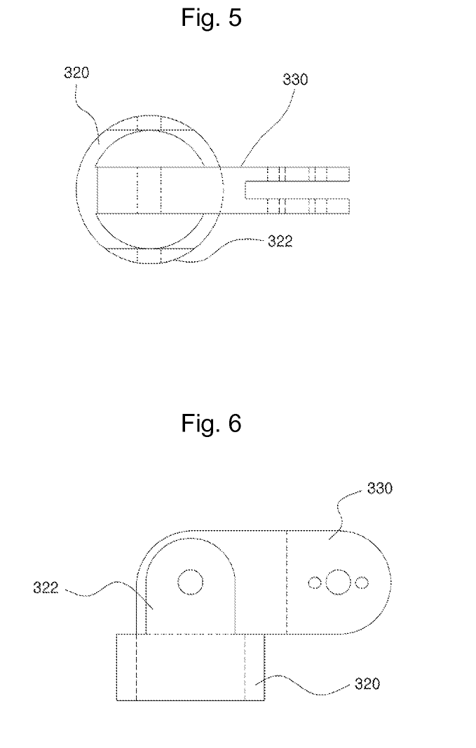

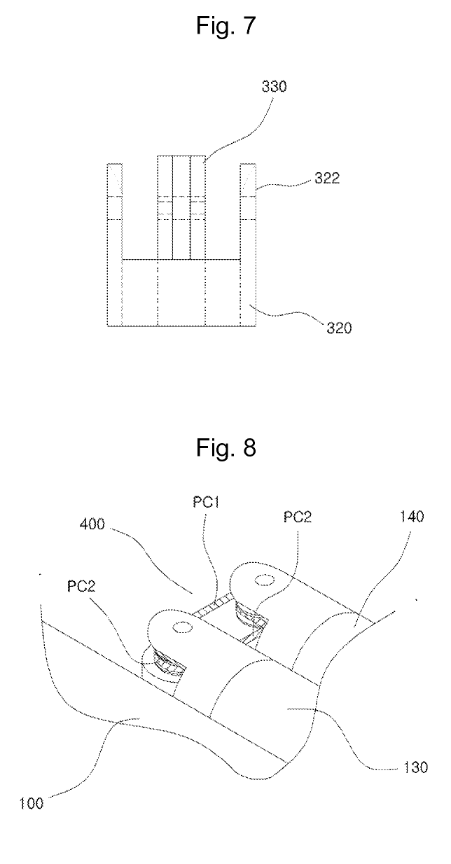

The first control main body 330 is then positioned between the first and the second connection end pairs 312 and 322. The first control main body 330 has a connection hole 332 at its end to receive a predetermined rotation axis, so the first control main body 330 can join with the first connection end 312 through the rotation axis. Further, the other end of the first control main body 330 can be integrally formed at the inside of the second connection ring 320 (of course, the end of the first control main body 330 may be coupled to the second connection ring 320 by other fastening elements. Also, other control main bodies to be described later can also be coupled to corresponding connection rings by any other suitable fastening element, without necessarily being integrated with any other connection rings as one unit). In relation to this, FIGS. 5, 6, and 7 provide the configuration of the first control main body 330 to be integrally formed with the second connection ring 320, which is seen from different angles.

Returning to FIG. 4, two pairs of connection pulleys are positioned on the rotation axis of each end of the first control main body 330, as shown. As illustrated in the drawing, those pulley pairs are collectively referred to as first and second connection pulleys 340 and 350, respectively. The first and the second connection pulleys 340 and 350 may be arranged inside the first and the second connection end pairs 312 and 322, respectively, and may rotate independently of each other. Preferably, the first and the second connection pulleys 340 and 350 are of equal diameter and approximately twice as wide as the width of the first and the second pitch cables PC1 and PC2.

Moreover, a first yaw cable pulley 360 may be additionally positioned on the rotation axis of the pair of the first connection ends 312. The first yaw cable pulley 360 is secured to the first control main body 330 by means of a pair of fixing pins, so it operates following the operation of the first control main body 330. It has substantially the same width as a yaw cable to be mentioned later.

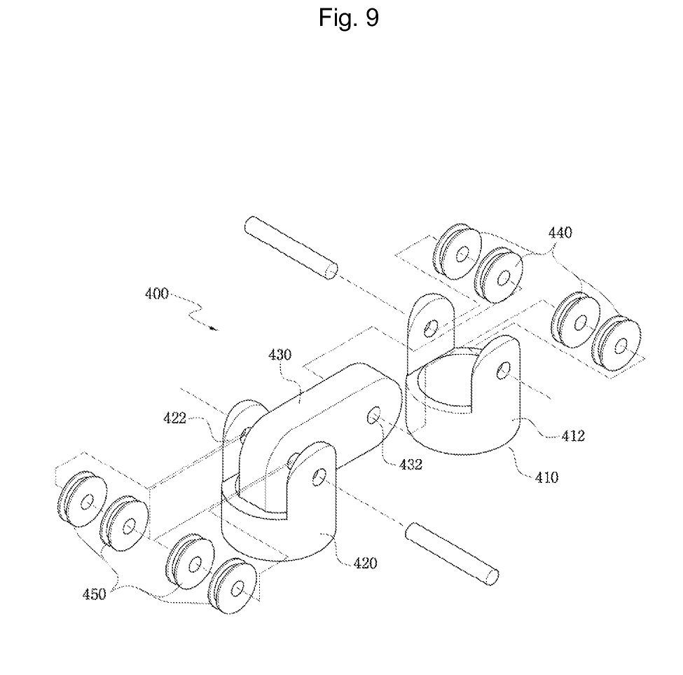

FIG. 8 is a detailed view of ‘b’ portion in FIG. 1, which shows that the first control shaft 130 and the second control shaft 140 are connected by means of a second yaw control part 400, in accordance with the first embodiment of the present invention. FIG. 9 is an exploded perspective view showing the configuration of the second yaw control part 400 in FIG. 8. The second yaw control part 400 has substantially the same configuration as the first yaw control part 300 except that there is no separate yaw cable pulley positioned at a second control main body 430, so details thereon will be omitted here for simplicity.

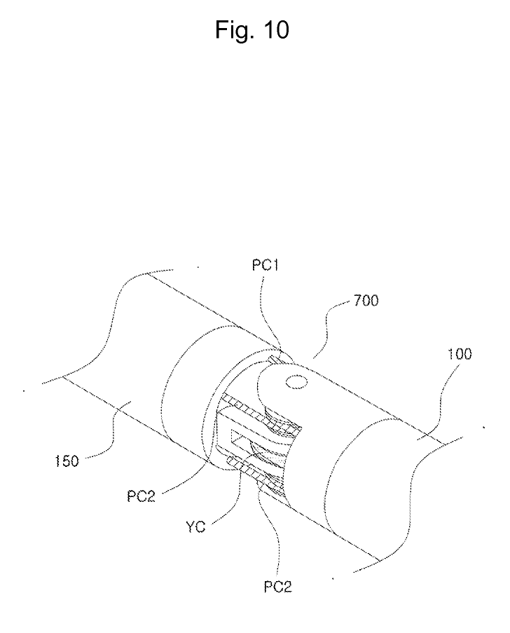

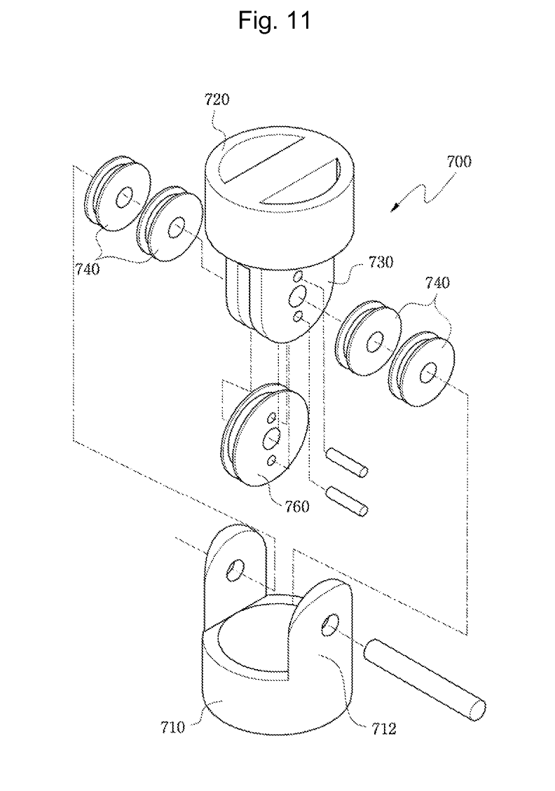

FIG. 10 is a detailed view of ‘c’ portion in FIG. 1, which shows that the main shaft 100 and the first actuating shaft 150 are connected by means of a first yaw control part 700, in accordance with the first embodiment of the present invention. FIG. 11 is an exploded perspective view showing the configuration of the first yaw actuating part 700, in which a first actuating main body 730 composed of two plates spaced apart by a predetermined distance is integrally formed with a second connection ring 720 and a pair of connection ends 712 extended from a first connection ring 710 are positioned on a rotation axis.

Here, a second yaw cable pulley 760 can be fixedly positioned in a space between the two plates that constitute the first actuating main body 730, and two pairs of connection pulleys 740 can be positioned near either end of the first actuating main body 730. At this time, the pairs of connection pulleys 740 are installed to be able to rotate independently of each other.

The second yaw cable pulley 760 has substantially the same width as a yaw cable YC (to be described later), and each of the connection pulleys 740 is approximately twice as wide as the first and the second pitch cables PC1 and PC2. Also, it is preferable to make the first yaw cable pulley 360 and the second yaw cable pulley 760 have substantially the same diameter as each other.

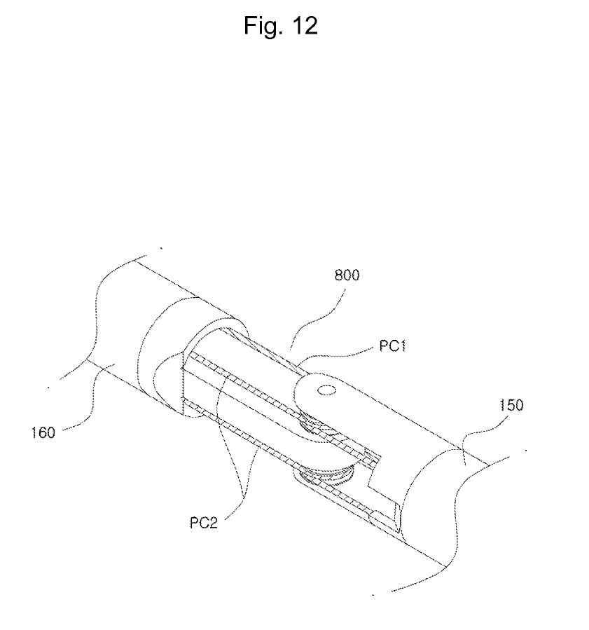

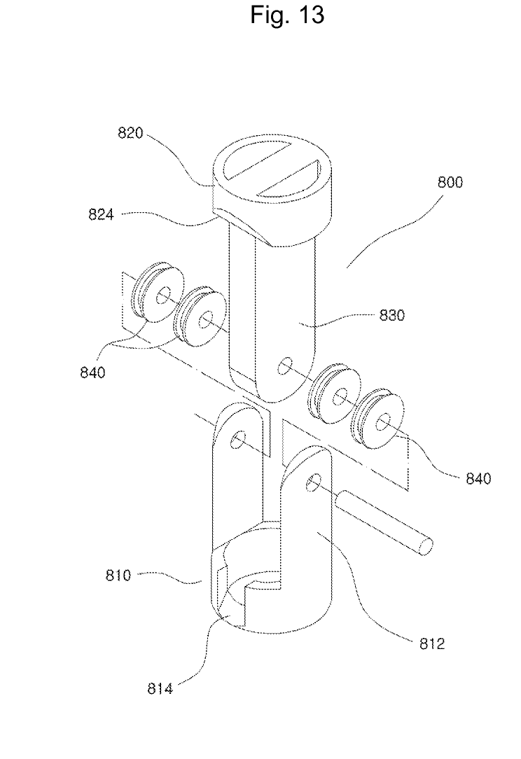

FIG. 12 is a detailed view of ‘d’ portion in FIG. 1, which shows that the first actuating shaft 150 and the second actuating shaft 160 are connected by a second yaw actuating part 800, in accordance with the first embodiment of the present invention, and FIG. 13 is an exploded perspective view showing the configuration of the second yaw actuating part 800.

As can be seen from the drawings, the second yaw actuating part 800 has a similar configuration to that of the first yaw actuating part 700, except that a pair of first connection ends 812 extended from the first connection ring 810 and a second actuating main body 830 integrally formed with a second connecting ring 820 are relatively longer, so as to increase the operation range of the first actuating shaft 150 and the second actuating shaft 160 to 90 degrees or more with respect to each other and that it does not have a yaw cable pulley. Thus, further details on such a configuration will not be provided here.

Additionally, it is preferable that an expansion groove 814 may be included in part of the first connection ring 810 to increase the rotation range of the second actuating main body 830. Also, a predetermined corner rounding portion 824 as shown in FIG. 13 may be included in part of the second connection ring 820.

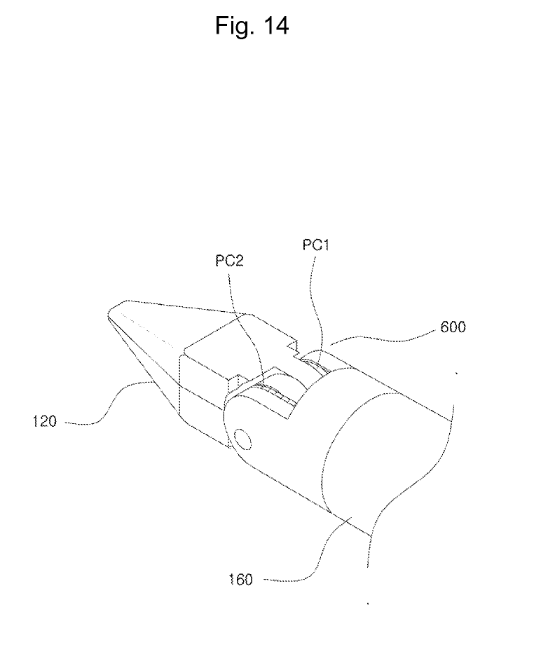

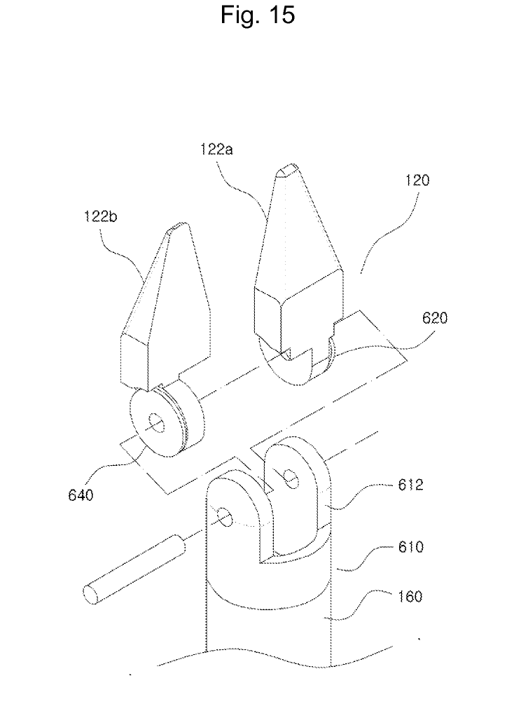

FIG. 14 is a detailed view of ‘e’ portion in FIG. 1, which shows that the second actuating shaft 160 and the end effector 120 are connected by a pitch actuating part 600. FIG. 15 is an exploded perspective view showing a connection between the end effector 120 and the second actuating shaft 160 in accordance with the first embodiment of the present invention. Referring to these drawings, the end effector 120 is composed of a first rod 122a and a second rod 122b each of which has a truncated pyramid shaped end and connected to each other with a rotation axis. The end effector 120 is connected to the second actuating shaft 160 by a first pitch cable pulley 620 and a second pitch cable pulley 640, that belong to the pitch cable pulley 600 and are positioned at the end of the first and the second rods 122a and 122b, respectively. At this time, the rotation axis may place across a pair of connection ends 612 extended from a connection ring 610 at one end of the second actuating shaft 160.

The first pitch cable pulley 620 and the second pitch cable pulley 640 are connected by the first and the second pitch cables PC1 and PC2 (to be described later) to operate following the operation of the pitch control part 200. Accordingly, the first and the second rods 122a and 122b that constitute the end effector 120 can operate independently of each other.

Meanwhile, the first pitch cable pulley 620 and the second pitch cable pulley 640 have substantially the same diameter and substantially the same width as the first and the second pitch cables PC1 and PC2. In addition, it is preferable that the first and the second pitch cable pulleys 620 and 640 that constitute the pitch actuating part 600 have substantially the same diameter as the first and the second pitch cable pulleys 220 and 240 that constitute the pitch control part 200.

Meanwhile, as the first and the second rods 122a and 122b that constitute the end effector 120 operate independently of each other, the end effector 120 is open or closed. Therefore, the end effector 120 may be utilized for a tool, i.e., a clamp, a grasper, scissors, a stapler, a needle holder, or the like, which is used to do the surgery inside the body. If necessary, unlike the one shown in the drawing, the end effector 120 in accordance with another embodiment of the present invention may be any element, such as a hook electrode, which does not need to be opened or closed.

In the following, a connection state of the first and the second pitch cables PC1 and PC2 and the yaw cable YC will be explained in detail, with reference to corresponding drawings, together with the drawings discussed above.

First, a connection state of the yaw cable YC will be explained.

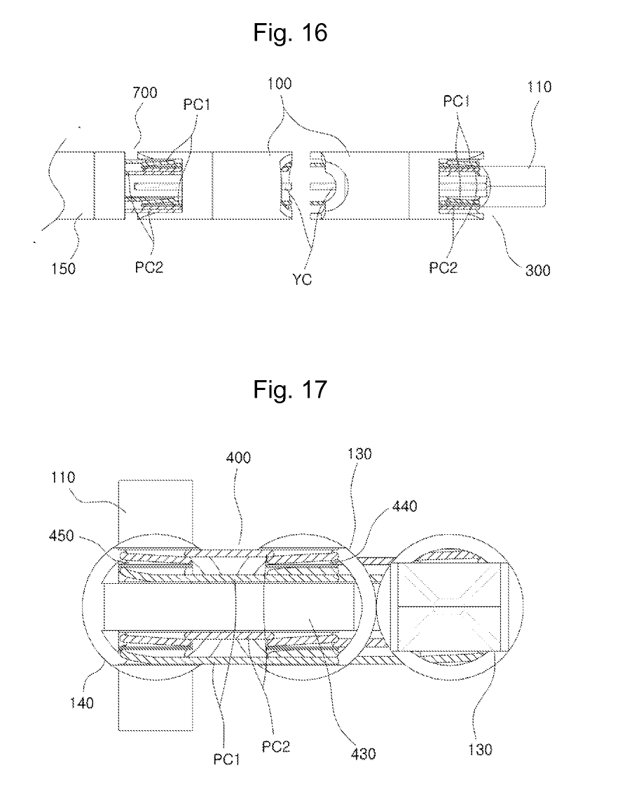

As depicted in FIG. 16, the yaw cable YC is wound around the first yaw cable pulley 360 and the second yaw cable pulley 760 that are positioned in the first control main body 330 and the first actuating main body 730, respectively, wherein the first control main body 330 and the first actuating main body 730 respectively constitute the first yaw control part 300 and the first yaw actuating part 700 connected to both ends of the main shaft 100. Thus, a motion of the first control shaft 130 in a yaw direction can be transferred to the first actuating shaft 150.

In accordance with a preferred embodiment of the present invention, the yaw cable YC can be wound around in a manner that the first yaw cable pulley 360 and the second yaw cable pulley 760 may rotate in the same direction, but, depending on user's needs, it may be wound in an elongated ‘8’ shape (this may be equally applied to winding of other cables) to make the first yaw cable pulley 360 and the second yaw cable pulley 760 rotate in opposite directions from each other. Either way, the yaw cable YC is wound passing through the inner space of the shaft 100. Also, if the first yaw cable pulley 360 and the second yaw cable pulley 760 have substantially the same diameter, the first control shaft 130 and the first actuating shaft 150 move substantially to the same amount.

Referring again to FIG. 3, it can be seen that the first pitch cable pulley 220 and the second pitch cable pulley 240, which together constitute the pitch control part 200 connecting the second control shaft 140 to the adjustment handle 110, are wound with the first pitch cable PC1 and the second pitch cable PC2, respectively.

Referring now to FIG. 17 that shows the cable connection in the second yaw control part 400 in accordance with the first embodiment of the present invention, the first and the second pitch cables PC1 and PC2 are respectively wound around the first and the second connection pulleys 440 and 450 that are included in the second yaw control part 400. In detail, two pairs of the first connection pulleys 440 and two pairs of the second connection pulleys 450 are positioned on either end of the second control main body 430 of the second yaw control part 400, and the first and second pitch cables PC1 and PC2 each are wound around the first and the second connection pulleys 440 and 450 with the second control main body 430 between them, as illustrated. At this time, as shown in FIG. 17, although the first pitch cable PC1 is practically wound around the first connection pulley 440, it may not be so for the second connection pulley 450. On the other hand, the second pitch cable PC2 can be substantially wound around both the first and the second connection pulleys 440 and 450. Similar to the examples discussed above, the cables in the following examples may also be wound in any form as long as they are not released from the pulleys or entangled with each other to impede the operation.

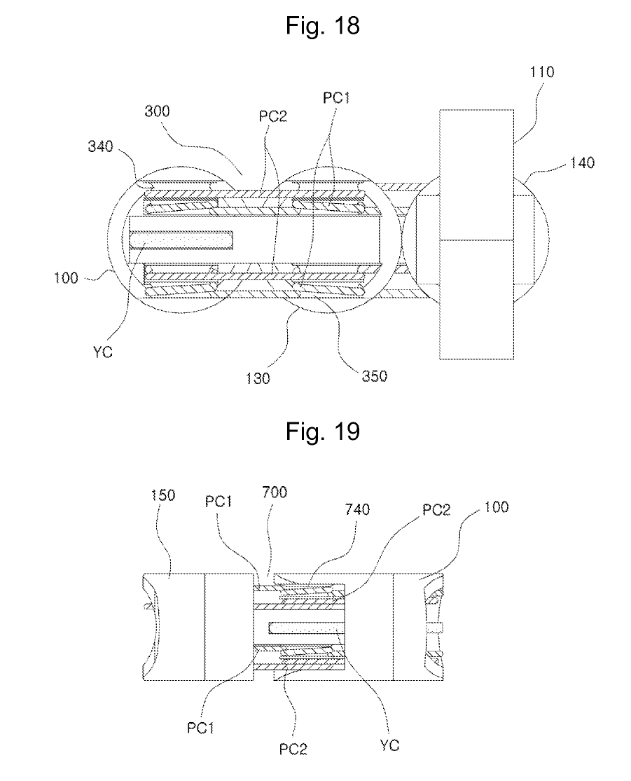

FIG. 18 shows an example of how cables are connected in the first yaw control part in accordance with the first embodiment of the present invention.

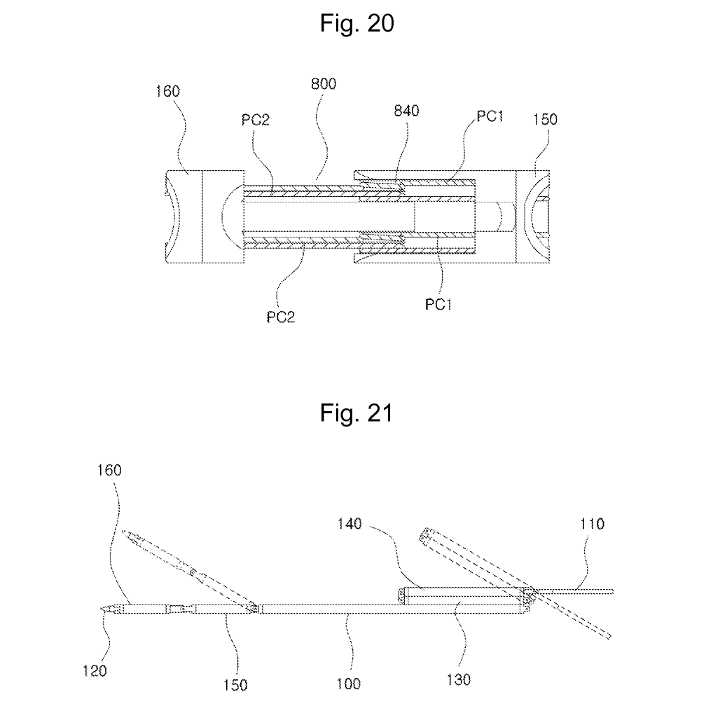

As depicted in FIGS. 16 and 19, the first and the second pitch cables PC1 and PC2 can be connected to the first yaw actuating part 700 from the first yaw control part 300 to the first yaw actuating part 700 by way of the shaft 100. In addition, the first and the second pitch cables PC1 and PC2 are wound around the connection pulley 740 of the first yaw actuating part 700 and further, as shown in FIG. 20, around the connection pulley 840 of the second yaw actuating part 800. Alternatively, the first and the second pitch cables PC1 and PC2 may be wound around the first and the second pitch cable pulleys 620 and 640 of the pitch actuating part 600. This configuration is already discussed with reference to FIG. 14.

Now, the operation of the tool for minimally invasive surgery as configured above in accordance with the first embodiment of the present invention will be explained in detail.

First, a user may arrange the tool for minimally invasive surgery as shown in FIG. 1. Next, the user puts his or her hand in the enclosure 112 of the adjustment handle 110 that is installed at one end of the tool 1 for minimally invasive surgery and holds the adjustment handle 110.

Hereinafter, it is assumed that (+) and (−) motions in the pitch direction designate motions in the upper and lower sides about the user, respectively, for convenience of explanation about the operation of the adjustment handle 110 in the pitch direction. Similarly, it is assumed that (+) and (−) motions in the yaw direction designate motions in the right and left sides about the user, respectively, for convenience of explanation about the operation of the adjustment handle 110 in the yaw direction.

As shown in FIG. 21, when the user holding the adjustment handle 110 rotates the adjustment handle 110 to the left, the first and the second control shafts 130 and 140 rotate with respect to the rotation axis of the first yaw cable pulley 360 of the first yaw control part 300. Here, since the first yaw cable pulley 360 is secured to the first control main body 330 of the first yaw control part 300, it engagedly rotates with the first and the second control shafts 130 and 140. This rotating motion can be transferred to the second yaw cable pulley 760 of the first yaw actuating part 700 by the yaw cable YC that is connected to the first yaw cable pulley 360.

Because the second yaw cable pulley 760 of the first yaw actuating part 700 is also fixed to the first actuating main body 730, the first actuating shaft 150 moves in the same direction and to substantially the same angle as the first and the second control shafts 130 and 140. At this time, the second actuating shaft 160 engagedly rotates with the first actuating shaft 150 as shown in the drawing.

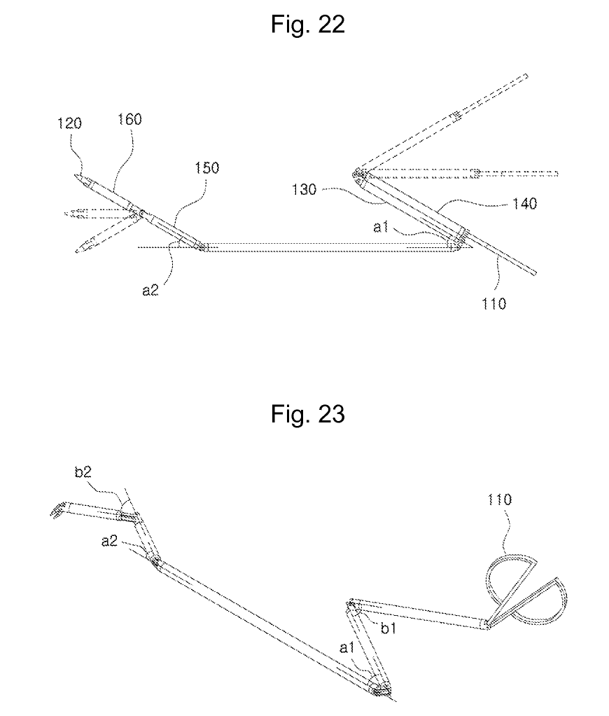

Referring now to FIG. 22, when the user rotates the adjustment handle 110 to the right, the second control shaft 140 can rotate with respect to the rotation axis of the first connection pulley 440 of the second yaw control part 400. At this time, the second connection pulley 450 of the second yaw control part 400 does not really rotate, so the second control main body 430, like the second control shaft 140, can rotate with respect to the rotation axis of the first connection pulley 440. What happens in this procedure, according to the drawing, the first pitch cable PC1 is pushed towards the first control shaft 130 from the handle adjustment 110, and the second pitch cable PC2 is pulled towards the adjustment handle 110 from the first control shaft 130.

Thereafter, when the user continues to rotate the adjustment handle 110 to the right, the second control shaft 140 and the second actuating shaft 160 can move as shown in FIG. 23. In this case, because an angle a2 is substantially the same as an angle a1 and an angle b2 is substantially the same as an angle b1, the user can conveniently make the end effector 120 face a direction he or she wants merely by operating the adjustment handle 110.

Meanwhile, as depicted in FIG. 23, the user may cause the end effector 120 to move in a pitch direction or to open/close, merely by operating the adjustment handle 110. Referring again to the drawings discussed earlier, the first pitch cable PC1 transfers a motion of the first pitch cable pulley 220 of the adjustment handle 110 to the first pitch cable pulley 620, via the first and the second connection pulleys 440 and 450, the first and the second connection pulleys 340 and 350, and the connection pulleys 740 and 840, thereby operating the first rod 122a of the end effector 120. And, the second pitch cable PC2 transfers a motion of the second pitch cable pulley 240 of the adjustment handle 110 to the second pitch cable pulley 640, via the first and the second connection pulleys 440 and 450, the first and the second connection pulleys 340 and 350, and the connection pulleys 740 and 840, thereby operating the second rod 122b of the end effector 120. In result, when the first rod 112a that is connected to the first pitch cable pulley 220 and the second rod 112b that is connected to the second pitch cable pulley 240 rotate upwardly together, the end effector 120 moves downward. Moreover, when the user operates the adjustment handle 110 in a certain way to increase or decrease an angular distance between the first and the second rods 112a and 112b of the end effector 120 in the pitch direction, the angular distance between the first and the second rods 112a and 112b in the pitch direction increases or decreases accordingly, thereby allowing the end effector 120 to open or close.

For better understanding, the operation of the minimally invasive surgical tool in accordance with the first embodiment of the present invention has been explained in order of the operation in the yaw direction, the operation in the pitch direction, and the opening/closing of the end effector for convenience of explanation, but it may be performed in a different order from the one mentioned above, or two or more operations may be performed at the same time. Either way, the same operation results are obtained based on the operating principle as discussed above (this may be equally applied to the following embodiments).

Meanwhile, if all of the pulleys used for the control parts and the actuating parts are of the same size, the displacement amount of the adjustment handle 110 and the displacement amount of the end effector 120 are also same. That is, the adjustment handle 110 and the end effector 120 will move to different amounts, provided that different sized pulleys are used for the control parts and the actuating parts. For instance, if the first yaw cable pulley 360 is larger in diameter than the second yaw cable pulley 760, the second yaw cable pulley 760 rotates at a greater angle than the first yaw cable pulley 360 under the yaw direction control by the user. As a result, the first actuating shaft 150 can rotate further than the first control shaft 130.

### Embodiment 2

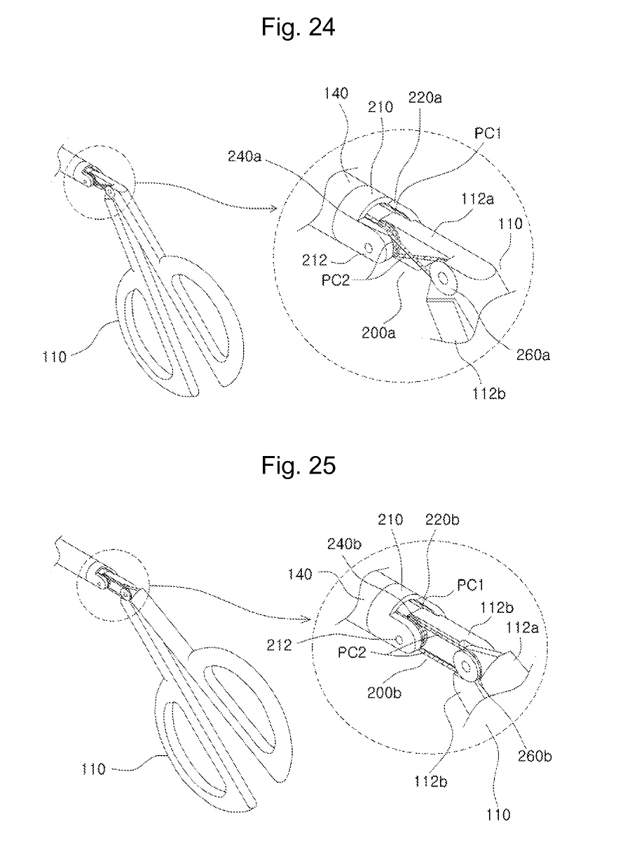

FIG. 24 is a perspective view showing a connection between an adjustment handle 110 and a second control shaft 140 of a tool for minimally invasive surgery in accordance with a second embodiment of the present invention. In accordance with the second embodiment of the present invention, the adjustment handle 110 is connected to the second control shaft 140 by means of a pitch control part 200a.

More details on such a configuration will be given below.

The pitch control part 200a can include a first pitch cable pulley 220a, a second pitch cable pulley 240a, and a third pitch cable pulley 260a. As shown in the drawing, out of first and second rods 112a and 112b that constitute the adjustment handle 110, the first rod 112a has the first pitch cable pulley 220a fixed to its extended end and the second pitch cable pulley 240a positioned on the same rotation axis to rotate independently inside a connection end 212 that is formed on a connection ring 210 at the end of the second control shaft 140. Further, a third pitch cable pulley 260a can be positioned on the rotation axis to which the first and the second rods 112a and 112b are connected. Here, the third pitch cable pulley 260 operates in conjunction with the second rod 112b as shown.

Next, a first pitch cable PC1 is connected to the first pitch cable pulley 220a, and a second pitch cable PC2 connected to the second pitch cable pulley 240a is extendedly connected further to the third pitch cable pulley 260a. Preferably, the second pitch cable pulley 240a is approximately three times wider than the second pitch cable PC2. Meanwhile, the first pitch cable pulley 220a, the second pitch cable pulley 240a, and the third pitch cable pulley 260a have substantially the same width.

With the above configuration, a user is able to control a motion of an end effector 120 in a pitch direction merely by operating the adjustment handle 110, or open/close the end effector 120 by operating only the second pitch cable PC2 which is achieved by operating only the second rod 112b connected to the third pitch cable pulley 260a.

To help the user get a better understanding on the opening/closing operation of the end effector 120, in this embodiment, the second pitch cable PC2 is preferably wound around in an elongated 8 shape between the second pitch cable pulley 240a and the third pitch cable pulley 260a. Referring to FIG. 25, an end of the second rod 112b of the adjustment handle 110 is extended, and the first pitch cable pulley 220b and the second pitch cable pulley 240b may be positioned near either side of the extended end. In this case, the user may operate the adjustment handle 110 in a way to cause the first and the second rods 112a and 112b operate together in a pitch direction to thus control the operation of the end effector 120 in the pitch direction, or control the opening/closing operation of the end effector 120 by changing an angular distance of the first rod 112a to the second rod 112b.

Besides what has been explained so far, the tool for minimally invasive surgery in accordance with the second embodiment of the present invention is identical to the first embodiment in its configuration and operation, so detailed description on them will be omitted. It is also obvious that the configuration of the tool in accordance with the second embedment of the present invention can be applied to the following fourth and fifth embodiments.

### Embodiment 3

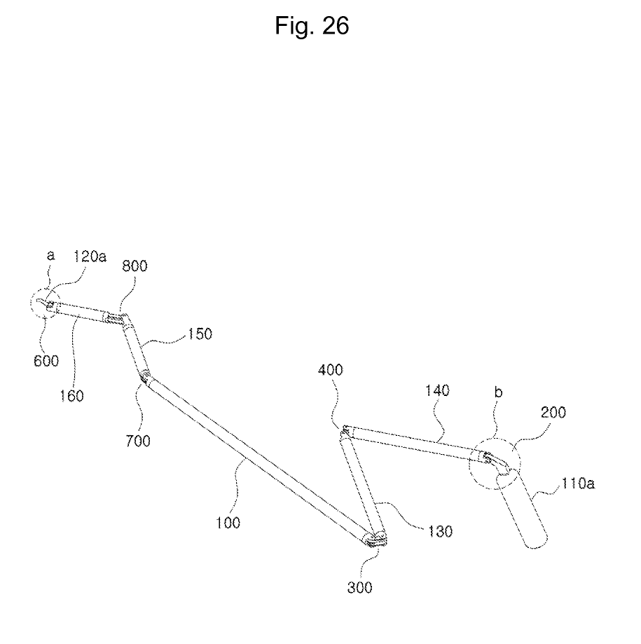

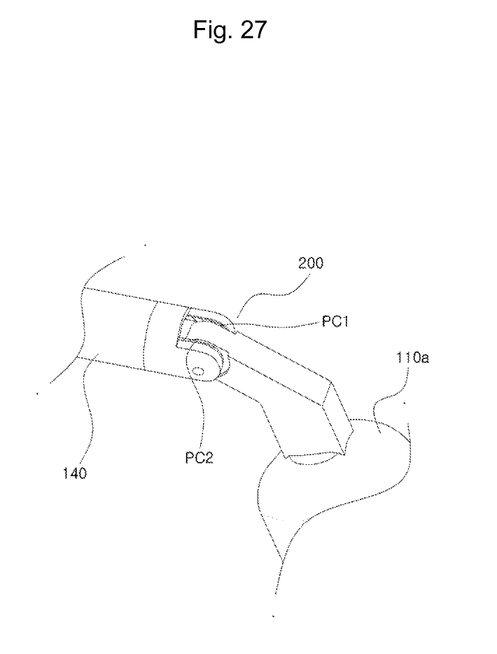

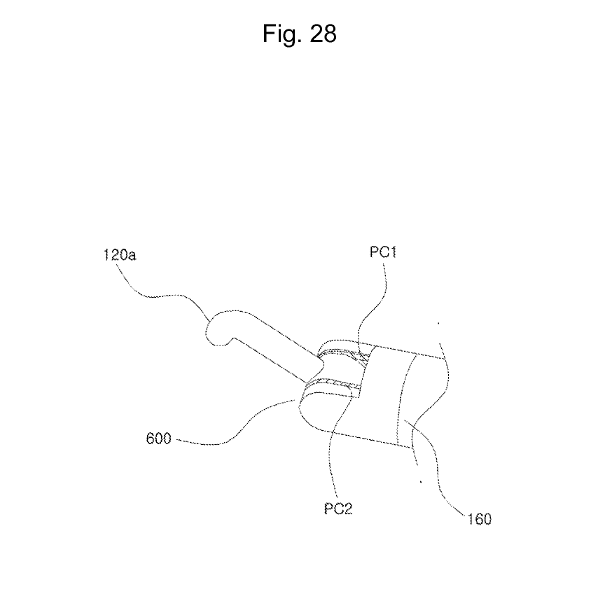

FIG. 26 is a perspective view showing the outer appearance of a tool for minimally invasive surgery in accordance with a third embodiment of the present invention, FIG. 27 is a detailed view of ‘b’ portion in FIG. 26, and FIG. 28 is a detailed view of ‘a’ portion in FIG. 26.

In accordance with the third embodiment of the present invention, an adjustment handle 110a for controlling an operation of an end effector 120a in a hook electrode form is connected to a second control shaft 140 by means of a pitch control part 200, and the end effector 120a is connected to a second actuating shaft 160 by means of a pitch actuating part 600.

In this embodiment, first and second pitch cables PC1 and PC2 operate together to transfer a motion of the adjustment handle 110a in a pitch/yaw direction to the end effector 120a.

In addition, unlike in the first embodiment, the end effector 120a of this embodiment has a bar shape with bendable portions (or any other shape, e.g., a ring shape, depending on user's needs as long as the opening/closing operations are not accompanied).

The tool for minimally invasive surgery of this embodiment basically has the same configuration as that of the first embodiment except that it does not have a mechanism for opening/closing the end effector. So details on the configuration will be omitted here for simplicity. The technical aspects of this embodiment may be equally applied to the following embodiments.

### Embodiment 4

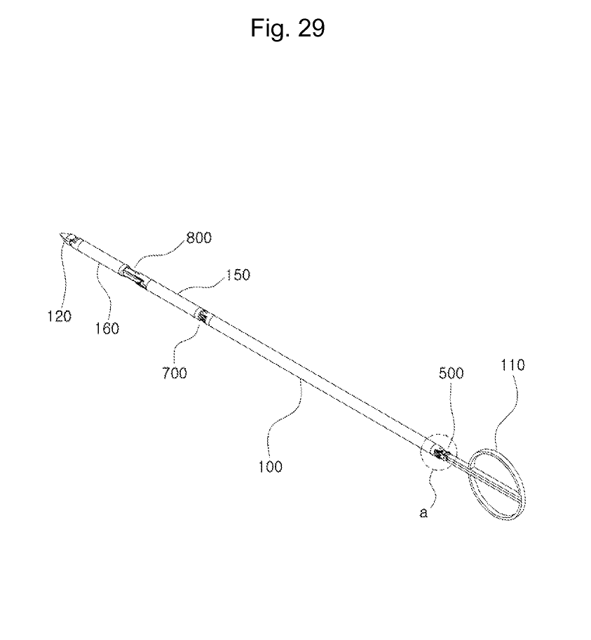

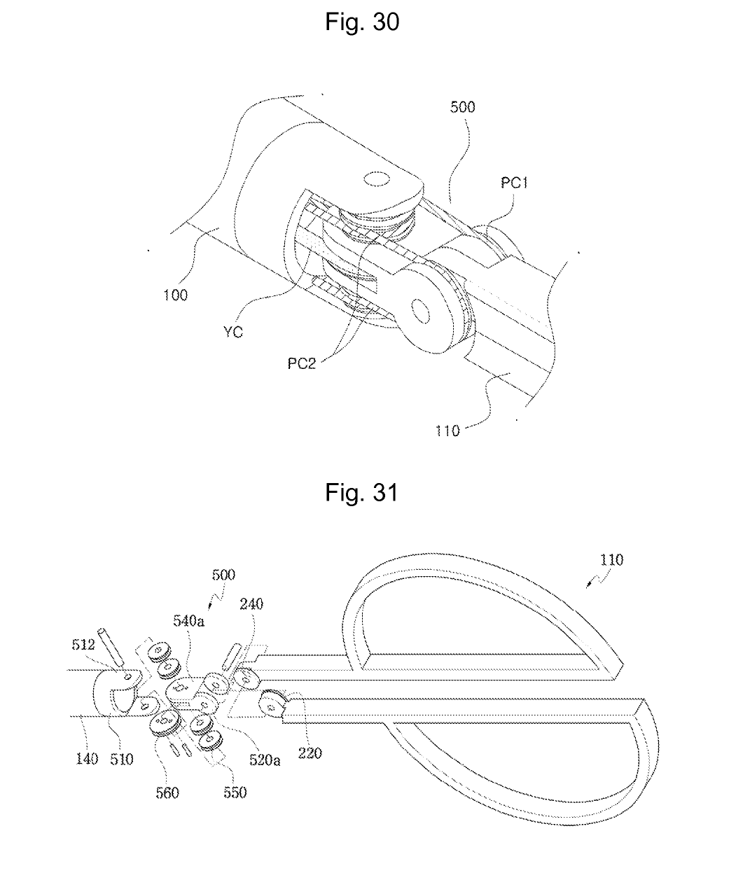

FIG. 29 is a perspective view showing the outer appearance of a tool for minimally invasive surgery in accordance with a fourth embodiment of the present invention, FIG. 30 is a detailed view of ‘a’ portion in FIG. 29, and FIG. 31 is an exploded perspective view showing the configuration of a connection part 500 between a shaft 100 and an adjustment handle 110 in accordance with the fourth embodiment of the present invention.

As depicted in FIGS. 29 and 30, the adjustment handle 110 can be connected directly to a main shaft 100, and the connection part 500, where first and second pitch cables PC1 and PC2 and a yaw cable YC all pass through, is provided between the main shaft 100 and the adjustment handle 110. Besides, as can be see from FIG. 29, the minimally invasive surgical tool of this embodiment basically has a configuration similar to that of the first embedment.

Now, a configuration of the connection part 500 will be explained.

Referring to FIG. 31, the connection part 500 can be configured in a manner that a pair of pitch connection ends 520a is composed of two circular plates spaced apart by a predetermined distance, and a pair of yaw connection ends 540a is composed of two plates spaced apart by a predetermined distance and arranged orthogonally to the pair of pitch connection ends 520a. A first pitch cable pulley 220 and a second pitch cable pulley 240 are positioned inside the pair of the pitch connection ends 520a in such a way that they can rotate with respect to the rotation axis where first and second rods 112a and 112b constituting the adjustment handle 110 are connected. In addition, a yaw cable pulley 560 is fixedly positioned inside the pair of the yaw connection ends 540a. The first pitch cable pulley 220 and the second pitch cable pulley 240 have the same width as the first and the second pitch cables PC1 and PC2, and the yaw cable pulley 560 has the same width as the yaw cable YC.

Further, two pairs of connection pulleys 550 are rotatably positioned on either side of the pair of yaw connection ends 540a. At this time, even though the yaw cable pulley 560 and the connection pulleys 550 are coaxially positioned, they can rotate independently of each other. Preferably, each of the connection pulleys 550 is approximately twice as wide as the pitch cables PC1 and PC2 to be described later.

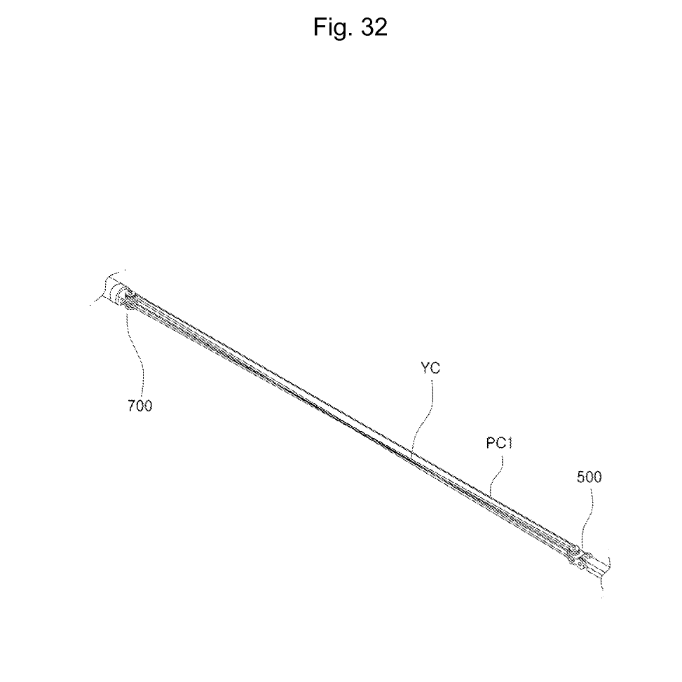

The connection pulleys 550 of the connection part with the above configuration are connected to a second yaw cable pulley 750 of a first yaw actuating part 700 by means of the yaw cable YC. Here, a connection state of the yaw cable may be in an elongated 8 shape as shown in FIG. 32.

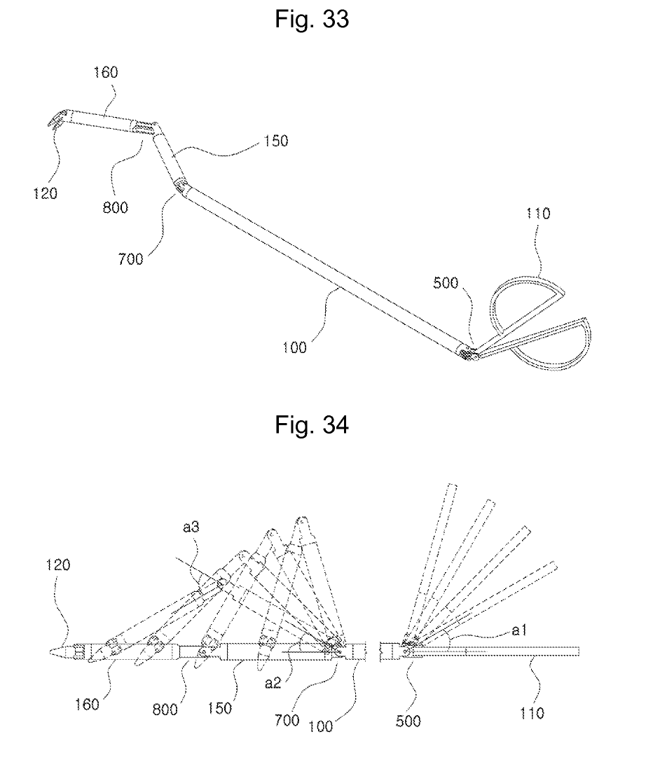

The minimally invasive surgical tool in accordance with the fourth embodiment of the present invention as set forth above enables a user to operate the adjustment handle 110 as shown in FIG. 33 to control the end effector 120 in pitch and/or yaw direction, and/or to open or close the end effector 120 by increasing or decreasing an angular distance between two rods that constitute the adjustment handle 110.

That is to say, since the yaw cable YC is connected in an elongated ‘8’ shape as depicted in FIG. 32, when the user operates the adjustment handle 110, one side of the yaw cable YC is pulled while the other side thereof is pushed. Then, the first actuating shaft 150 rotates about the first yaw actuating part 700 as depicted in FIG. 34. At this time, an angle a2 is substantially the same as a rotation angle a1 of the adjustment handle 110 by the yaw cable YC.

A motion of the adjustment handle 110 in the yaw direction is transferred not only by the yaw cable YC but also by the first and the second pitch cables PC1 and PC2. However, the first and the second pitch cables PC1 and PC2 transfer motions of the adjustment handle 110 in the yaw direction to a second yaw actuating part 800 and in the opposite direction to that of the motion transferred by the yaw cable YC. In result, when the user rotates the adjustment handle 110 only to the angle a1, the second actuating shaft 800 and the end effector 120 rotate only to an angle a3 shown. Ideally, the angle a3 may be twice the angle a1.

As explained so far, unlike the first embodiment, the fourth embodiment of the present invention requires no separate control shaft.

### Embodiment 5

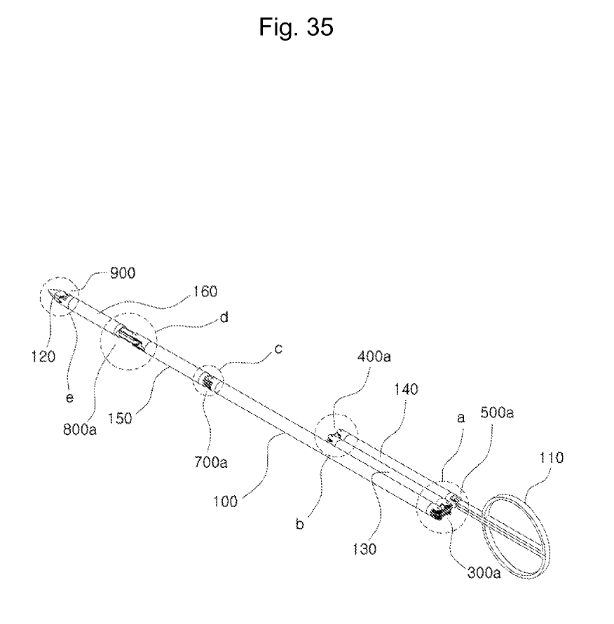

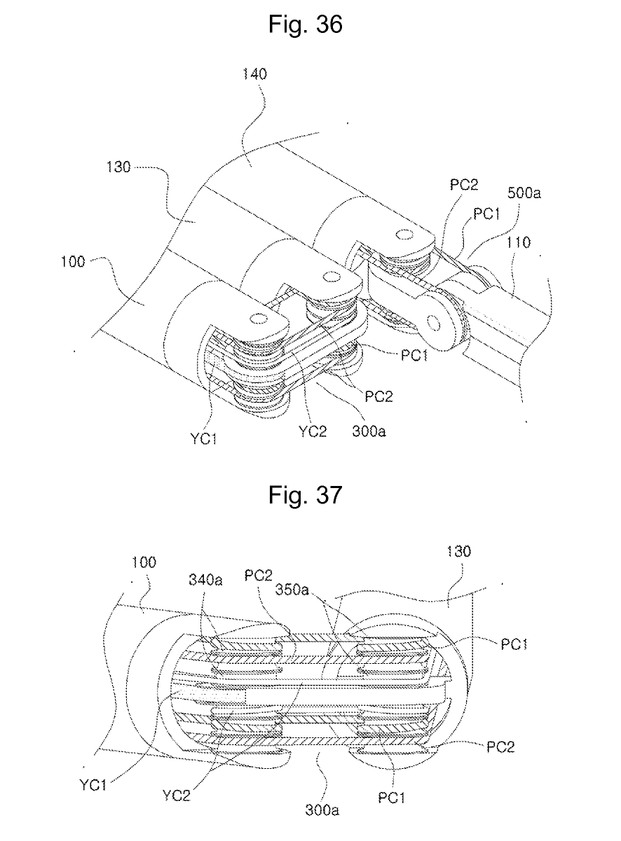

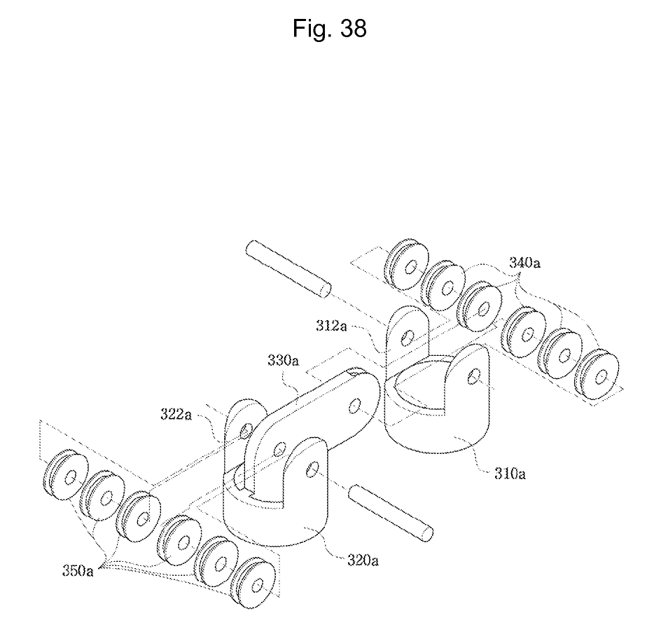

FIG. 35 is a perspective view showing the outer appearance of a tool for minimally invasive surgery in accordance with a fifth embodiment of the present invention. Referring to FIG. 35, the minimally invasive surgical tool in accordance with the fifth embodiment of the present invention, similar to that of the first embodiment, basically includes a main shaft 100, an adjustment handle 110, an end effector 120, first and second control shafts 130 and 140, first and second actuating shafts 150 and 160, first and second yaw control parts 300a and 400a, and first and second yaw actuating parts 700a and 800a. In addition to them, the tool further includes a first connection part 500a and a second connection part 900 to be discussed later. More details about the configuration will be provided below. FIG. 36 is a detailed view of ‘a’ portion in FIG. 35. To elaborate the configuration of the first yaw control part 300a in FIG. 36, FIG. 37 presents another view of the first yaw control part 300a seen from a different angle. And, FIG. 38 is an exploded perspective view showing the configuration of the first yaw control part 300a in accordance with the fifth embodiment of the present invention.

The configuration of the first yaw control part 300a of the fifth embodiment is similar to the configuration of the first yaw control part 300 of the first embodiment except that three pairs of the first connection pulleys 340a and three pairs of the second connection pulleys 350a are positioned near either end of a first control main body 330a. Referring to FIGS. 36 and 37, it can be seen how the first and the second pitch cables PC1 and PC2 and the first and the second yaw cables YC1 and YC2 are wound around each of the first and the second connection pulleys 340a and 350a of the first yaw control part 300a. Here, each of the first and the second connection pulleys 340a and 350a can be approximately twice as wide as the first and the second pitch cables PC1 and PC2, and the second yaw cables YC2.

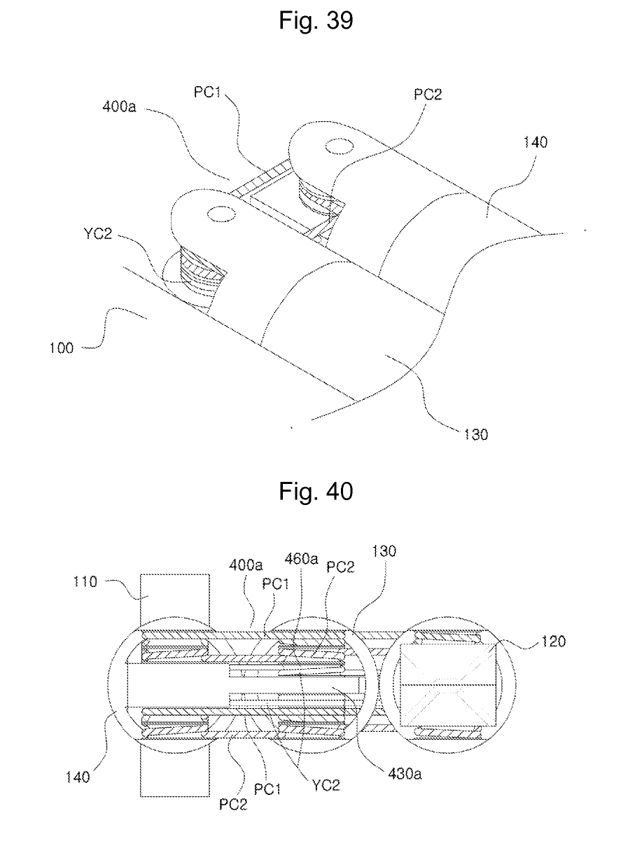

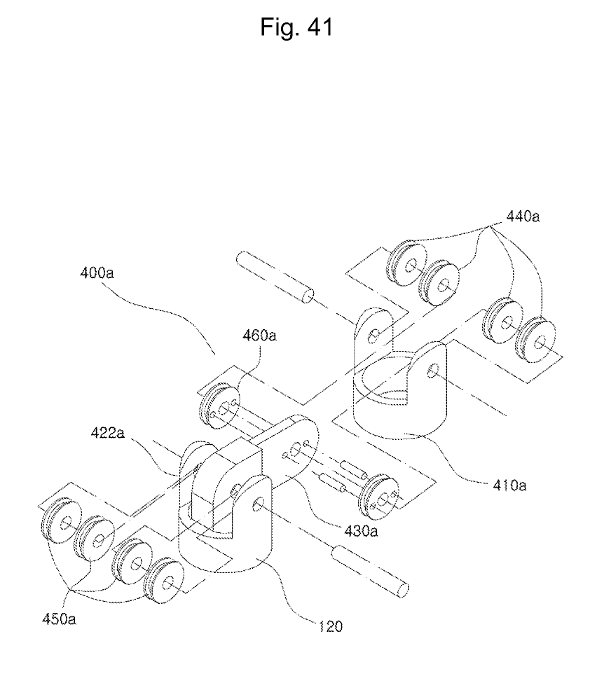

FIG. 39 is a detailed view of ‘b’ portion in FIG. 18. To elaborate the configuration of the second yaw control part 400a in FIG. 39, FIG. 40 presents another view of the second yaw control part 400a seen from a different angle. And, FIG. 41 is an exploded perspective view showing the configuration of the second yaw control part 400a.

The configuration of the second yaw control part 400a is similar to the configuration of the second yaw control part 400 of the first embodiment except that the third connection pulleys 460a are further fixedly positioned at one end of a second control main body 430a. Referring to FIG. 40, it can be seen how the first and the second pitch cables PC1 and PC2 and the second yaw cables YC2 are wound around each of the first, the second and the third connection pulleys 440a, 450a and 460a of the second yaw control part 400a. Here, each of the first, the second and the third connection pulleys 440a, 450a and 460a is approximately twice as wide as the first and the second pitch cables PC1 and PC2, and the second yaw cables YC2.

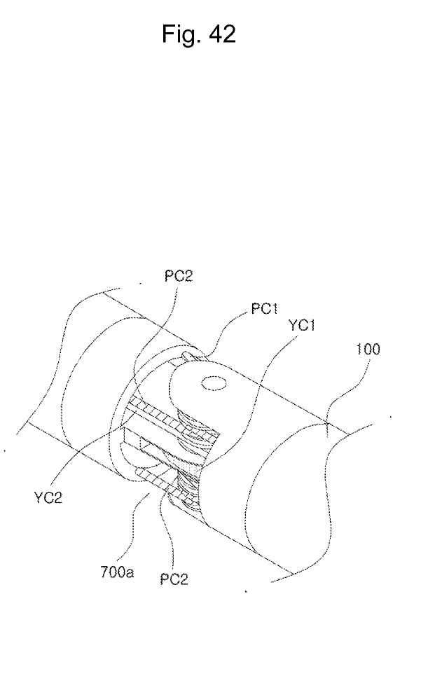

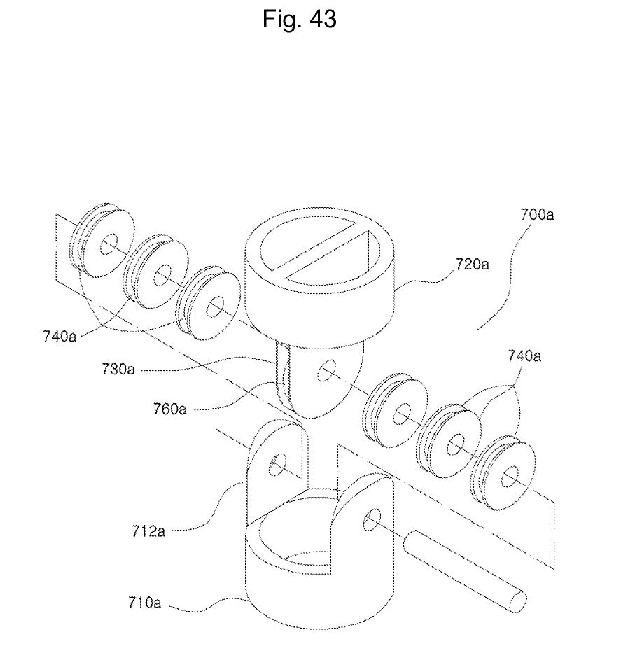

FIG. 42 is a detailed view of ‘c’ portion in FIG. 35, and FIG. 43 is an exploded perspective view showing the configuration of the first yaw actuating part 700a in accordance with the fifth embodiment of the present invention.

Since the configuration of the first yaw actuating part 700a is substantially the same as the configuration of the first yaw actuating part 700 in the first embodiment, except that three connection pulleys 740a are provided to each side of a first actuating main body 730a and that the second yaw cables YC2 are connected to the innermost connection pulley 740a out of three connection pulleys 740a. Therefore, detailed description on the configuration will be omitted here for brevity.

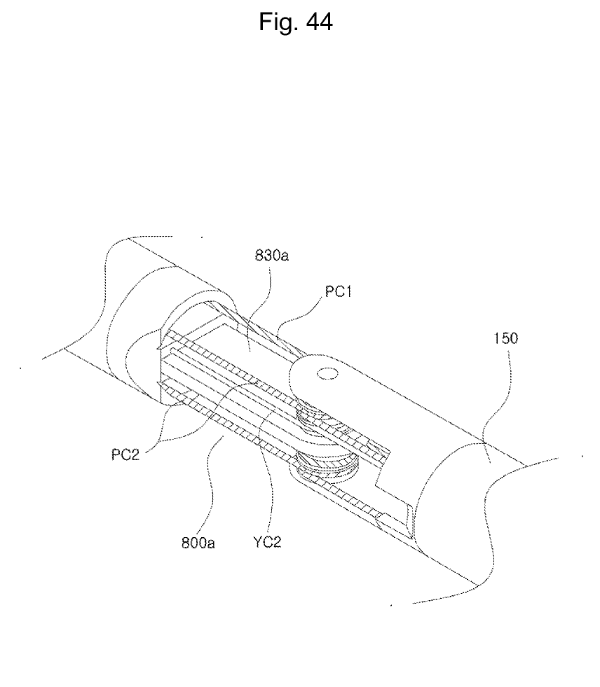

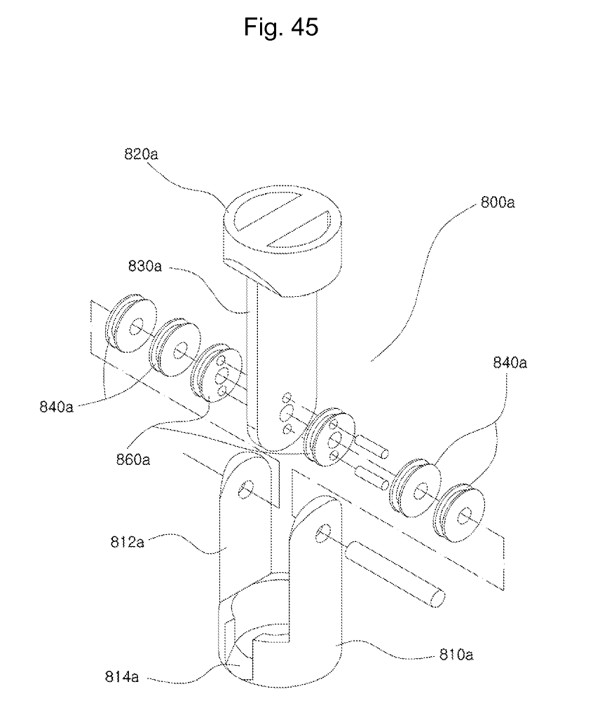

FIG. 44 is a detailed view of ‘d’ portion in FIG. 35, and FIG. 45 is an exploded perspective view showing the configuration of the second yaw actuating part 800a in accordance with the fifth embodiment of the present invention.

In the second yaw actuating part 800a, two pairs of first connection pulleys 840a are positioned on either side of a second actuating main body 830a, and a second connection pulley 860a to which the second yaw cables YC2 are connected is positioned between the first connection pulley 840a and the second actuating main body 830a. Here, the second connection pulley 860a to which the second yaw cables YC2 are connected is fixed to the second actuating main body 830a. Also, the second yaw cables YC2 are connectively fixed to the second actuating main body 830a.

The following is a further explanation about a connection state of the second yaw cables YC2 in accordance with this embodiment, which is not found in the first embodiment. As shown in FIG. 40, one end of the second yaw cables YC2 can be connectively fixed to the second control main body 430a. Also, the second yaw cables YC2 are wound around the third connection pulley 460a of the second yaw control part 400a, and connected to the first and the second yaw actuating parts 700a and 800a in order, via the innermost pulley out of the first and the second connection pulleys 340a and 350a included in the first yaw control part 300a, as shown in FIG. 37. The other end of the second yaw cables YC2 are connectively fixed to the second actuating main body 830a, as shown in FIG. 44.

Hereinafter, an operation of the tool for minimally invasive surgery in accordance with the fifth embodiment will be described in detail.

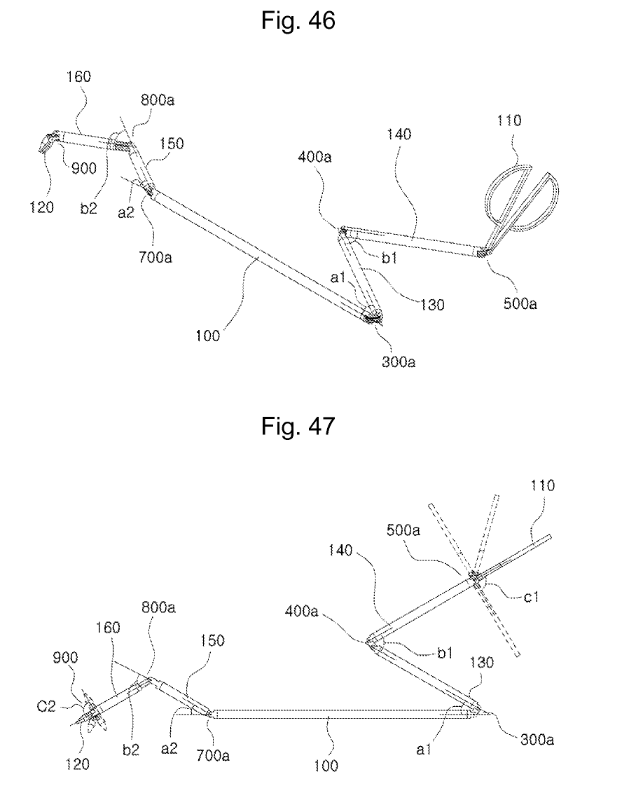

In this embodiment, the first yaw cable YC1, like the yaw cable YC in the first embodiment, is positioned to connect the first yaw control part 300a and the first yaw actuating part 700a, thereby transferring a motion of the first yaw control part 300a in the yaw direction to the first yaw actuating part 700a. In result, as shown in FIG. 46, the first actuating shaft 150 operates following the operation of the first control shaft 130 and the angles a1 and a2 obtained at this time are substantially identical to each other. In this case, not only the first yaw cable YC1 but also the first and the second pitch cables PC1 and PC2 and the second yaw cables YC2 affect motions of the first yaw control part 300a and the first yaw actuating part 700a in the yaw direction. However, if the operations of the first and the second pitch cables PC1 and PC2 and the second yaw cables YC2 in the yaw direction are not triggered by the operation of the first yaw control part 300a, the operation of the first yaw actuating part 700a is hardly affected by the effect of the first yaw cable YC1.

In addition, as depicted in FIGS. 39 and 40, the first and the second pitch cables PC1 and PC2 and the second yaw cables YC2 are positioned at the second yaw control part 400a to transfer a motion of the second yaw control part 400a in the yaw direction to the second yaw actuating part 800a. At this time, as depicted in FIG. 46, an angle b2 between the first actuating shaft 150 and the second actuating shaft 160 is substantially identical to an angle b1 between the first control shaft 130 and the second control shaft 140. In this case, the first and the second pitch cables PC1 and PC2 operate according to the same operation principle of the second yaw control part 400 and the second yaw actuating part 800 in the first embodiment, and the second yaw cables YC2, together with the first and the second pitch cables PC1 and PC2, transfers a motion of the second yaw control part 400a in the yaw direction to the second yaw actuating part 800a. Further, the second yaw cables YC2 serve to prevent motions of the second yaw control part 400a and the second yaw actuating part 800a in the yaw direction from being influenced by the yaw-direction operations of the first and the second pitch cables PC1 and PC2 in the first connection part 500a to be described later.

As shown in FIG. 36, the first connection part 500a in the fifth embodiment can be configured similarly to the connection part 500 in the fourth embodiment, such that it helps the first and the second pitch cables PC1 and PC2 operate in both pitch and yaw directions. In fact, the first connection part 500a is configured as illustrated in FIG. 30, except that no yaw cable YC is connected thereto. Therefore, when the user rotates the adjustment handle 110 to operate the first connection part 500a, its motions in the pitch/yaw direction because of that are transferred to the second connection part 900 through the first and the second pitch cables PC1 and PC2, thereby determining an operation direction of the end effector 120 (where the second connection part 900 has substantially the same configuration as the first connection part 500a). In this case, as shown in FIG. 47, the user may rotate the adjustment handle 110 to wider angles with respect to the second control shaft 140, and the motion of the first connection part 500a following the rotation of the adjustment handle 110 is transferred to the second connection part 900, thereby allowing the end effector 120 to operate to a wider range. Referring to FIG. 47, if the user rotates the adjustment handle 110 to the left, it causes the end effector 120 to rotate to the right. Thus, an angle c2 between the end effector 120 and the second actuating shaft 160 can be identical to a rotation angle c1 of the adjustment handle 110 to the left.

The following is a detailed explanation about specific application examples of the present invention, which are achieved by employing at least one of the above embodiments or by adopting such application examples to at least one of the embodiments.

### Application Example 1

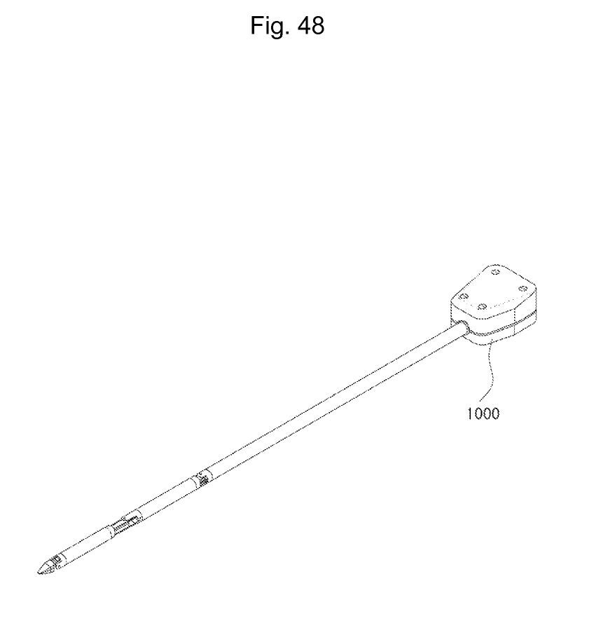

FIG. 48 is a perspective view showing the outer appearance of a tool for minimally invasive surgery in accordance with the present invention, which shows that a controller 1000 performing functions of the adjustment handle 110 and the first and the second control shafts 130 and 140 in several embodiments is connected to one end of a shaft 100.

Here, the controller 1000 can be electrically controlled by an electromotive means such as a motor to make a motion in a pitch/yaw direction and opening/closing operations as the adjustment handle 110 of the previous embodiments has done.

Any person skilled in the art can freely take a configuration for the controller 1000 by applying conventional electric drive control techniques. Some exemplary configurations for the controller 1000 can be found in related arts, U.S. Pat. No. 4,853,874 entitled “Master-slave Manipulator with Scaling”, U.S. Pat. No. 5,779,623 entitled “Positioner for Medical Instruments”, and U.S. Pat. No. 6,102,850 entitled “Medical Robotic System”.

However, it should be understood that these specific related arts are mentioned merely for illustrative purposes, not for limiting the configuration of the controller 1000 of the present invention in any intentional way.

### Application Example 2

Under the present invention, particularly, under the fifth embodiment of the present invention, the degree of freedom in a yaw direction is excessively large that a surgery may be impeded by that. To resolve this, as disclosed in Korean Patent Application No. 2008-79126 by the same applicant, a B/F nut may be fastened to a bolt outside of a first control shaft 130, and a curved guide having one end being secured onto a shaft 100 and the other end being bolted may be installed. By doing so, a displacement pattern of the first yaw control part 300 is properly fixed, and further a displacement pattern of a first actuating part 700 is fixed, thereby making an additional control in the yaw direction using the other yaw control parts.

In relation to this application example, it should be understood that such elements that are installed at the shaft 100 and the first control shaft 130 may also be installed at the first control shaft 130 and the second control shaft 140, and that any other elements may be employed as long as they can restrict the motion of any of the shafts.

### Application Example 3

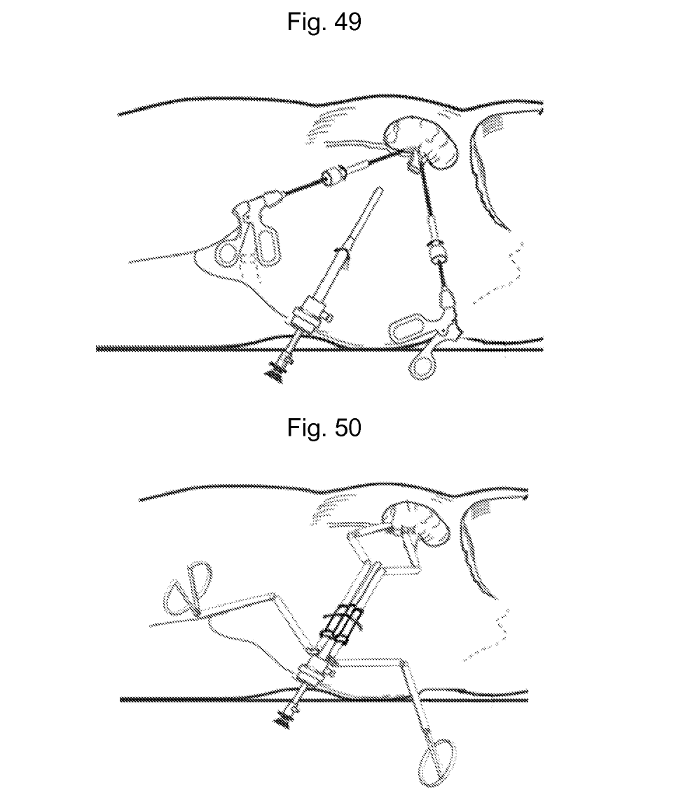

Hereinafter, examples of how to utilize the minimally invasive surgical tool of the present invention that has been taught in easy-to-understand manner through the embodiments set forth above will be introduced with reference to FIGS. 49 and 50.

First, according to one application example of the present invention, it takes only one incision for surgery, contrarily to forming plural incisions as shown in FIG. 49 in a patient's body. FIG. 50 illustrates a case where two tools for minimally invasive surgery of the present invention are inserted in parallel through only one incision to perform a surgery. In this case, it is preferable that the two tools for minimally invasive surgery are provided to perform symmetrical motions to each other, as shown. That is, a surgeon may hold a tool in each hand and perform a surgery. Optionally, an endoscope may be additionally inserted through one incision as shown in FIG. 45 (one of benefits of this case is that a parallel arrangement between the endoscope and the surgical tool is easily secured, so the surgeon becomes aware of his or her action more intuitively).

Needless to say, the method for using the minimally invasive surgical tool in accordance with the present invention is not limited to the one discussed above. For example, an endoscope and one tool for minimally invasive surgery of the present invention may be inserted through one incision for surgery to let them stay side by side, or another tool of related art may be further inserted through one incision for surgery while the endoscope and one tool for minimally invasive surgery of the present invention have been inserted through the same incision to stay side by side.

The two minimally invasive surgical tools can be arranged in parallel and perform symmetrical motions because the first and the second control main bodies 330 and 430 that constitute the first and the second yaw control parts 300 and 400 and the second yaw actuating part of the present invention are arranged orthogonally to the first and the second control shafts 130 and 140, such that one can move the tools without causing any collision between instruments.

Moreover, it is not necessary to set a limit to the number of surgical tools to be inserted through one incision, and wide variety of surgical tools can be freely used as technical advances bring lighter, smaller, and finer surgical tools.

### Application Example 4

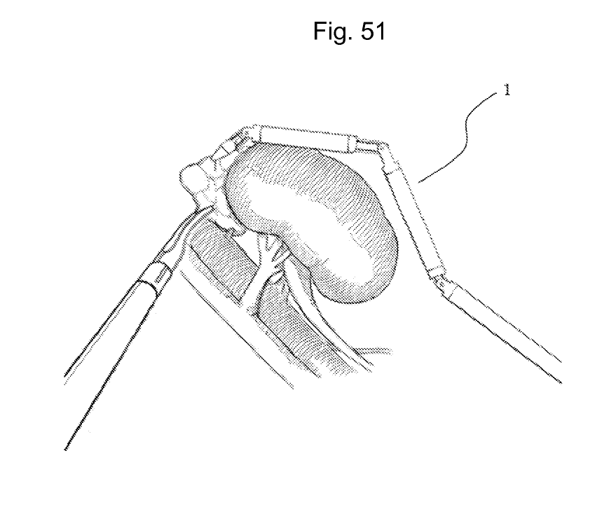

Referring to FIG. 51, a more simple and intuitive example will be helpful to understand advantages of the present invention. FIG. 51 is an exemplary view showing that the tool of the present invention can access relatively easily to an adrenal gland passing by the kidney which is one of organs in a patient's body. That is, using the minimally invasive surgical tool of the present invention can make high-degree-of-freedom motion to perform a required or needed operation, by easily avoiding an organ in the way without a limitation to the position of an incision.

In accordance with the present invention having the configurations as above, the following remarkable effects can be achieved.

1. In accordance with the present invention, provided is a minimally invasive surgical tool, the end effector of which operates corresponding to the operations in pitch and yaw directions and/or the opening and closing operations from an adjustment handle. Further, in accordance with the present invention, provided is a minimally invasive surgical tool, the end effector of which can operate with greater degrees of freedom than in accordance with the conventional technique.

2. In accordance with the present invention, provided is a minimally invasive surgical tool that can be freely controlled by a user without any complicated control element. Further, in accordance with the present invention, provided is a minimally invasive surgical tool that can dexterously operate with a relatively simple drive control element.

3. In accordance with the present invention, provided is a minimally invasive surgical tool that has small volume and weight and may be easily moved.

4. In accordance with the present invention, provided is a minimally invasive surgical tool, which has a plurality of joint portions so that it can access an area hidden by a specific human organ for surgery.

5. In accordance with the present invention, provided is a minimally invasive surgical tool, which requires only a minimum number of incisions (preferably, only one incision) on a patient's body for surgery and still enables an elaborate and easy surgical operation.

6. In accordance with the present invention, provided is a minimally invasive surgical tool, which is more advanced than the minimally invasive surgical tool described in Korean Patent Application Nos. 2008-51248 and No. 2008-61894 previously filed by the present inventor.

7. In accordance with the present invention, provided is a novel method to use the minimally invasive surgical tool according to the present invention.

While the present invention has been described with respect to certain preferred embodiments, it will be apparent to those skilled in the art that various changes and modifications may be made without departing from the scope of the invention as defined in the following claims.

1. A tool for minimally invasive surgery comprising,
a main shaft,
a first control shaft and a second control shaft positioned in sequence from one end of the main shaft,
a first actuating shaft and a second actuating shaft positioned in sequence from the other end of the main shaft,
an adjustment handle positioned around one end of the second control shaft,
an end effector positioned around one end of the second actuating shaft,
a pitch control part positioned around one position of the positions between the main shaft and the first control shaft, between the first control shaft and the second control shaft, and between the second control shaft and the adjustment handle, for transferring a motion of the adjustment handle in a pitch direction to the end effector,
a first yaw control part and a second yaw control part positioned around the other positions of the positions between the main shaft and the first control shaft, between the first control shaft and the second control shaft, and between the second control shaft and the adjustment handle, respectively, for transferring a motion of the adjustment handle in a yaw direction to the end effector,
a pitch actuating part positioned around one position of the positions between the main shaft and the first actuating shaft, between the first actuating shaft and the second actuating shaft, and between the second actuating shaft and the end effector,
a first yaw actuating part and a second yaw actuating part positioned around the other positions of the positions between the main shaft and the first actuating shaft, between the first actuating shaft and the second actuating shaft, and between the second actuating shaft and the end effector, respectively,
a first pitch cable and a second pitch cable for transferring motions from the pitch control part, the first yaw control part, and the second yaw control part to the pitch actuating part, the first yaw actuating part, and the second yaw actuating part, respectively, and
a yaw cable for transferring a motion from the first yaw control part to the first yaw actuating part with the first pitch cable and the second pitch cable.

2. The tool as claimed in claim 1, wherein the adjustment handle and the end effector are configured to open and close, and the end effector is configured to be opened and closed by operations of the first pitch cable and/or the second pitch cable following an operation of the adjustment handle.

3. The tool as claimed in claim 1, wherein at least one space is formed inside at least one of the main shaft, the first control shaft, the second control shaft, the first actuating shaft, and the second actuating shaft.

4. The tool as claimed in claim 3, wherein at least a portion of the cables are positioned through at least part of the space.

5. The tool as claimed in claim 1, wherein at least one of the first yaw control part, the second yaw control part, the first yaw actuating part, and the second yaw actuating part comprises,
a control main body, and
a plurality of connection pulleys positioned near either end of the control main body,
wherein the each connection pulleys is positioned on a rotation axis of one end of the control main body.

6. The tool as claimed in claim 5, wherein the control main body is fixed to one of the two adjacent shafts.

7. The tool as claimed in claim 6, wherein the control main body is fixed at right angles substantially with the shaft.

8. The tool as claimed in claim 5, wherein at least one of the first yaw control part, the second yaw control part, the first yaw actuating part, and the second yaw actuating part further comprises a yaw cable pulley, the yaw cable pulley is connected to the yaw cable.

9. The tool as claimed in claim 1, wherein at least one of the pitch control part and the pitch actuating part comprises a first pitch cable pulley and a second pitch cable pulley, the first pitch cable pulley is connected to the first pitch cable, and the second pitch cable pulley is connected to the second pitch cable.

10. The tool as claimed in claim 9, wherein the pitch control part further comprises a third pitch cable pulley for actuating following an operation of one rod of the adjustment handle, and the third pitch cable pulley is connected to the first pitch cable or the second pitch cable.

11. A tool for minimally invasive surgery comprising,
a main shaft,
an adjustment handle positioned around one end of the main shaft,
a first actuating shaft and a second actuating shaft positioned in sequence from the other end of the main shaft,
an end effector positioned around one end of the second actuating shaft,
a connection part positioned between the main shaft and the adjustment handle for transferring motions of the adjustment handle in a pitch direction and a yaw direction to the end effector,
a pitch actuating part positioned around one position of the positions between the main shaft and the first actuating shaft, between the first actuating shaft and the second actuating shaft, and between the second actuating shaft and the end effector,
a first yaw actuating part and a second yaw actuating part positioned around the other positions of the positions between the main shaft and the first actuating shaft, between the first actuating shaft and the second actuating shaft, and between the second actuating shaft and the end effector, respectively,
a first pitch cable and a second pitch cable for transferring motions from the connection part to the pitch actuating part, the first yaw actuating part, and second yaw actuating part, and
a yaw cable for transferring a motion from the connection part to the first yaw actuating part with the first pitch cable and the second pitch cable.

12. The tool as claimed in claim 11, wherein the connection part comprises,
a pair of pitch connection ends,
a pair of yaw connection ends coupled to the pair of pitch connection ends substantially orthogonally,
a first pitch cable pulley and a second pitch cable pulley formed near the pair of pitch connection ends, and
a yaw cable pulley formed near the pair of yaw connection ends,
wherein the first pitch cable pulley is connected to the first pitch cable, the second pitch cable pulley is connected to the second pitch cable, and the yaw cable pulley is connected to the yaw cable.

13. The tool as claimed in claim 12, further comprising,
a plurality of connection pulleys positioned near the pair of yaw connection ends.

14. A tool for minimally invasive surgery comprising,
a main shaft,
a first control shaft and a second control shaft positioned in sequence from one end of the main shaft,
a first actuating shaft and a second actuating shaft positioned in sequence from the other end of the main shaft,
an adjustment handle positioned around one end of the second control shaft,
an end effector positioned around one end of the second actuating shaft,
a first connection part positioned between the second control shaft and the adjustment handle for transferring motions of the adjustment handle in a pitch direction and a yaw direction to the end effector,
a first yaw control part and a second yaw control part positioned between the main shaft and the first control shaft, and between the first control shaft and the second control shaft, respectively, for transferring a motion of the adjustment handle in a yaw direction to the end effector,
a second connection part positioned between the second actuating shaft and the end effector,
a first yaw actuating part and a second yaw actuating part positioned between the main shaft and the first actuating shaft, and between the first actuating shaft and second actuating shaft,
a first pitch cable and a second pitch cable for transferring motions from the first connection part, the first yaw control part, and the second yaw control part to the second connection part, the first yaw actuating part, and the yaw actuating part, respectively,
a first yaw cable for transferring a motion from the second yaw control part to the second yaw actuating part with the first pitch cable and the second pitch cable, and
a second yaw cable for transferring a motion from the first yaw control part to the first yaw actuating part with the first pitch cable, the second pitch cable, and the first yaw cable.

15. The tool as claimed in claim 14, wherein the adjustment handle and the end effector are configured to open and close, and the end effector is configured to be opened and closed by operations of the first pitch cable and/or the second pitch cable following an operation of the adjustment handle.

16. The tool as claimed in claim 14, wherein at least one space is formed inside at least one of the main shaft, the first control shaft, the second control shaft, the first actuating shaft, and the second actuating shaft.

17. The tool as claimed in claim 16, wherein at least a portion of the cables are positioned through at least part of the space.

18. The tool as claimed in claim 14, wherein at least one of the first yaw control part, the second yaw control part, the first yaw actuating part, and the second yaw actuating part comprises,
a control main body, and
a plurality of connection pulleys positioned near either end of the control main body,
wherein the each connection pulleys is positioned on a rotation axis of one end of the control main body.

19. The tool as claimed in claim 18, wherein the control main body is fixed to one of the two adjacent shafts.

20. The tool as claimed in claim 19, wherein the control main body is fixed at right angles substantially with the shaft.

21. The tool as claimed in claim 18, wherein at least one of the first yaw control part and the first yaw actuating part further comprises a yaw cable pulley, the yaw cable pulley is connected to the yaw cable.

22. The tool as claimed in claim 14, wherein the connection part comprised,
a pair of pitch connection ends,
a pair of yaw connection ends coupled to the pair of pitch connection ends substantially orthogonally, and
a first pitch cable pulley and a second pitch cable pulley formed near the pair of pitch connection ends,
wherein the first pitch cable pulley is connected to the first pitch cable, and the second pitch cable pulley is connected to the second pitch cable.

23. The tool as claimed in claim 22, further comprising,
a plurality of connection pulleys positioned near the pair of yaw connection ends.

24. The tool as claimed in claim 22, wherein the first connection part further comprises a third pitch cable pulley for actuating following an operation of one rod of the adjustment handle, and the third pitch cable pulley is connected to the first pitch cable or the second pitch cable.

25. A tool for minimally invasive surgery comprising,
a main shaft,
a first actuating shaft and a second actuating shaft positioned in sequence from one end of the main shaft,
a controller positioned around the other end of the main shaft,
an end effector positioned around one end of the second actuating shaft,
a pitch actuating part positioned around one position of the positions between the main shaft and the first actuating shaft, between the first actuating shaft and the second actuating shaft, and between the second actuating shaft and the end effector,
a first yaw actuating part and a second yaw actuating part positioned around the other positions of the positions between the main shaft and the first actuating shaft, between the first actuating shaft and the second actuating shaft, and between the second actuating shaft and the end effector, respectively, and
a first pitch cable, a second pitch cable, and a yaw cable for controlling operations of the pitch actuating part, the first yaw actuating part, and the second yaw actuating part,
wherein the controller comprises a pitch control module for controlling the pitch actuating part, a first yaw control module for controlling the first yaw actuating part, and a second yaw control module for controlling the second yaw actuating part.

26. A method for using a plurality of tools for minimally invasive surgery, wherein the plurality of tools includes the tool of claim 1, the tool of claim 11, and the tool of claim 14, the method comprising,
inserting a first tool of the plurality of tools for minimally invasive surgery through an incision of a patient's body,
wherein the first tool is selected from the group consisting of the tool of claim 1, the tool of claim 11, and the tool of claim 14.

27-28. (canceled)

29. The method of claim 26 further comprising,
inserting a second tool of the plurality of tools for minimally invasive surgery through an incision of a patient's body, wherein the second tool is selected from the group consisting of the tool of claim 1, the tool of claim 11, and the tool of claim 14.

30. The method of claim 29, wherein a range of motion of the first tool in a yaw direction is substantially symmetrical to a range of motion of the second tool in a yaw direction.

31. The method of claim 29, wherein the first tool and the second tool are inserted in parallel.
